(19) United States
(12) Patent Application Publication
(10) Pub. No.: US 2023/0117792 A1
(43) Pub. Date: Apr. 20, 2023

(54) USE OF SUBSTITUTED CINNAMIC ACID ESTERS AS STABILISERS FOR ORGANIC MATERIALS, STABILISED ORGANIC MATERIAL, METHOD FOR STABILISING ORGANIC MATERIALS AND SPECIFIC CINNAMIC ACID ESTERS

(71) Applicant: FRAUNHOFER-GESELLSCHAFT ZUR FÖRDERUNG DER ANGEWANDTEN FORSCHUNG E.V., München (DE)

(72) Inventors: Rudolf PFAENDNER, Darmstadt (DE); Jannik MAYER, Darmstadt (DE); Elke METZSCH-ZILLIGEN, Darmstadt (DE)

(73) Assignee: FRAUNHOFER-GESELLSCHAFT ZUR FÖRDERUNG DER ANGEWANDTEN FORSCHUNG E.V., München (DE)

(21) Appl. No.: 17/906,080

(22) PCT Filed: Mar. 19, 2021
(86) PCT No.: PCT/EP2021/057101

§ 371 Date: Sep. 12, 2022

(30) Foreign Application Priority Data
Mar. 27, 2020 (DE) 10 2020 203 987.9

(51) Int. Cl. C08K 5/526; C08K 5/372; C08K 5/103; C08K 5/00; C07C 69/734; C07C 69/736

(52) U.S. Cl. CPC C08K 5/526; C08K 5/372; C08K 5/103; C08K 5/005; C07C 69/734; C07C 69/736

(57) ABSTRACT

The present invention relates to the use of specific cinnamic acid esters as stabilizers of organic materials, in particular against oxidative, thermal and/or actinic degradation. The invention also relates to a corresponding stabilized organic material. The invention further relates to a method for stabilizing organic materials, in which specific cinnamic acid esters are incorporated into the organic material. According to the invention, specific new cinnamic acid esters are also disclosed.

The present invention relates to the use of specific cinnamic acid esters as stabilizers of organic materials, in particular against oxidative, thermal and/or actinic degradation. Furthermore, the present invention relates to a correspondingly stabilized organic material. A further aspect of the present invention relates to a method for stabilizing organic materials, in which specific cinnamic acid esters are incorporated into the organic material. According to the invention, specific new cinnamic acid esters are also disclosed.

Organic materials, such as plastics, are subject to aging processes, which ultimately lead to a loss of desired properties, such as e.g. mechanical characteristic values. This process, called autoxidation, leads to changes in the polymer chain, for example, in molecular weight or the formation of new chemical groups, arising from radical chain cleavages through mechanochemical processes or through UV radiation in the presence of oxygen. Stabilizers are therefore used to prevent or at least delay said aging. Important representatives of stabilizers are antioxidants, which interfere with the free radicals formed during autoxidation and thus interrupt the degradation process. A distinction is generally made between primary antioxidants, which can react directly with oxygen-comprising free radicals or C-radicals, and secondary antioxidants, which react with intermediately formed hydroperoxides (see C. Kröhnke et al. Antioxidants in Ullmann's encyclopedia of industrial chemistry, Wiley-VCH Verlag, Weinheim 2015). Typical representatives of primary antioxidants are, for example, phenolic antioxidants, amines, but also lactones. Classes of secondary antioxidants are phosphorus compounds, such as phosphites and phosphonites, but also organosulfur compounds, such as thioesters and disulfides. Usually, in practice, primary and secondary antioxidants are often combined, resulting in a synergistic effect.

Plastics formed from fossil raw materials such as petroleum or natural gas are increasingly being supplemented or replaced by plastics based on renewable raw materials obtained via biochemical processes. The question of sustainability then also arises for the primary and secondary antioxidants used therefor (and for plastics made from fossil raw materials). There is therefore a need for stabilizers based on renewable and available raw materials that are highly effective, have low volatility and are compatible with polymeric substrates.

Basically, primary antioxidants made from renewable raw materials, which are also occasionally used in plastics, are known. A typical example are tocopherols (vitamin E).

Tocopherols, similarly to conventional antioxidants, have a sterically hindered phenol structure and may be used alone or in combination with secondary antioxidants (for example S. Al-Malaika, Macromol. Symp. 2001, 176, 107-117). For example, tocopherols can be isolated from natural substances, such as e.g. wheat germ oil, sunflower oil or olive oil.

Further known phenols having an antioxidant effect in plastics are, for example, quercetin (B. Kirschweng et al., Eur. Pol. J. 2018, 103, 228-237), Dihydromyrecetin (B. Kirschweng et al., Pol. Degr. Stab. 2016, 133, 192-200), Derivate der Rosmarinsaure. (K. Doudin et al., Pol. Degr. Stab. 2016, 130, 126-134) or also tannin (W. J. Grigsby et al., Polymers 5 (2013) 344-360).

Furthermore, derivatives of ferulic acid (A. F. Reano et al. ACS Sustainable Chemistry and Engineering 4:6562-6571) and of caffeic acid (V. Ambrogi et al. Biomacromolecules 15:302-310) are also known.

However, most of the natural phenols require a great deal of effort when it comes to isolating, purifying or producing applicable secondary products.

This object is achieved with the features of patent claim 1 with regard to the use of certain cinnamic acid esters for stabilizing organic materials. The object is further achieved with a stabilized organic material according to patent claim 11 with regard to a method for stabilizing an organic material with the features of patent claim 14 and with regard to new cinnamic acid esters, which can be used as stabilizers for the purposes of the present invention, with the features of patent claim 15. The respective dependent claims represent advantageous developments.

Ferulic acid and its salts are used, for example, in the cosmetics industry or as pharmaceutical active ingredients (e.g. FR 2907338, CN 101181256, DE 1957433), the production of the salts being known in principle (e.g. AT 317184). Also known in the prior art are the stabilization of plastics with selected ester derivatives of ferulic acid (A. F. Reano et al. ACS Sustainable Chemistry and Engineering 4 (2015), 6562-6571, A. F. Reano et al. ACS Sustainable Chemistry and Engineering 3 (2015), 3486-3496) and of caffeic acid (V. Ambrogi et al. Biomacromolecules 15 (2014), 302-310).

However, said derivatives are produced by enzymatic syntheses in a relatively complex manner. Likewise known ferulic acid derivatives in the form of ester compounds are isosorbide esters (US 2007 189990), cholestanyl esters (WO 2018/153917) and oligomers and polymers of ferulic acid (US 2016 257846).

The object of the present invention was to provide sustainable antioxidants for plastics based on renewable raw materials with high effectiveness, high thermal stability and low volatility.

In a first aspect, the present invention thus relates to the use of a compound, or mixtures of a plurality of compounds, of the general Formula I

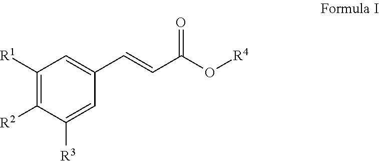

where

R¹, R² and R³ are each independently selected from the group consisting of hydroxy, linear or branched alkoxy groups having 1 to 6 carbon atoms and hydrogen, with the proviso that at least one of the moieties R¹, R² and R³ is a hydroxy moiety and/or a linear or branched alkoxy group having 1 to 6 carbon atoms, and

R⁴ is selected from the group consisting of a linear or branched alkyl moieties having at least 8 carbon atoms and the following moieties according to Formulas IIa, IIb and IIc,

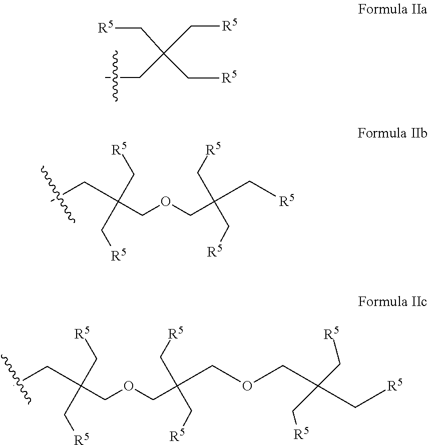

where R⁵ is the same or different at each occurrence and is selected from the group consisting of hydroxy and the following moiety according to Formula III,

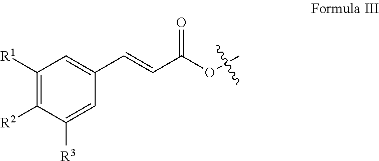

where R¹, R² and R³ are as defined above, for the stabilization of organic materials, in particular against oxidative, thermal and/or actinic degradation.

Surprisingly, it was found that the cinnamic acid derivatives used according to the invention can be used as new stabilizers based on renewable raw materials. A high level of effectiveness, environmental friendliness and a favorable cost structure compared to the stabilizers previously known from the prior art were found.

Plastics, coatings, lubricants, hydraulic oils, engine oils, turbine oils, gear oils, metalworking fluids, chemicals or monomers are particularly suitable as organic materials for the purposes of the present invention.

According to the invention, cosmetics are not counted to be among the organic materials.

According to a particularly preferred embodiment, the compound according to general Formula I is selected from the group consisting of the following compounds:

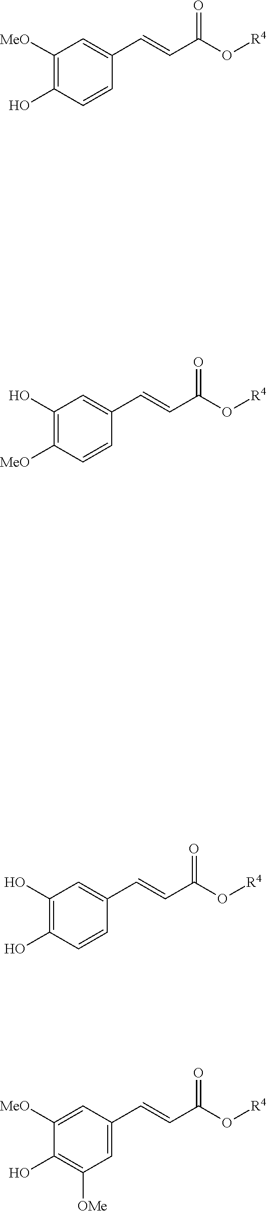

where R⁴ is as defined in claim 1.

For example, the linear or branched alkyl moiety having at least 8 carbon atoms of the moiety R⁴ can be derived from alcohols selected from the group consisting of octan-1-ol, nonan-1-ol, decan-1-ol, undecan-1-ol, lauryl alcohol, tridecane-1-ol, myristyl alcohol, cetyl alcohol, stearyl alcohol, ceryl alcohol, myricyl alcohol, palmitoleyl alcohol, oleyl alcohol, arachidyl alcohol, behenyl alcohol, erucyl alcohol, lignoceryl alcohol, montanyl alcohol, linoleyl alcohol, isotridecyl alcohol, geraniol, rhodinool, citronellol and cerol, particularly preferably lauryl alcohol and stearyl alcohol.

Preferred moieties R⁵ are selected from the group consisting of the following moieties.

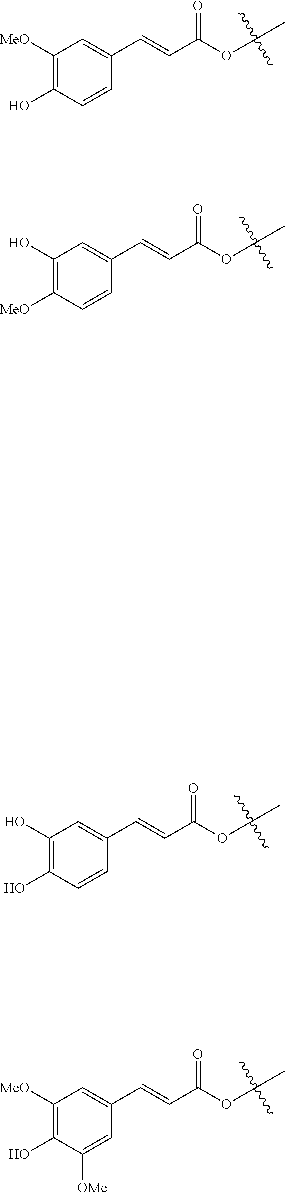 [SMILES: *OC(=O)/C=C/c1ccc(O)c(OC)c1 ; *OC(=O)/C=C/c1ccc(OC)c(O)c1 ; *OC(=O)/C=C/c1ccc(O)c(O)c1 ; *OC(=O)/C=C/c1cc(OC)c(O)c(OC)c1]

It is particularly preferred here that the substitution pattern of the moiety according to

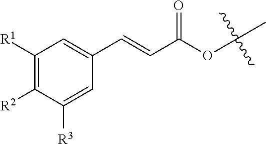

of the compound according to Formula I and the substitution pattern of the moiety R⁵ is identical.

This means that, for example, the substitution pattern of the moieties according to Formula IIa, IIb, IIc shown above with regard to R⁵ and the cinnamic acid moiety according to Formula I is identical. Such compounds are thus symmetrical.

Preferably, the compound according to general Formula I, or in the case of a mixture of a plurality of compounds according to general Formula I all of the compounds according to general Formula I, is/are present in a proportion by weight of from 0.01 to 10.00% by weight, preferably from 0.02 to 5.00% by weight, particularly preferably from 0.05 or 0.10 to 2.00% by weight, in the organic material.

According to a further preferred embodiment, the compound according to Formula I, or the mixtures thereof, is/are used to stabilize thermoplastic, elastomeric or duromer plastics. In particular, the plastic is selected from the group consisting of
a) polymers from olefins or diolefins, such as e.g. polyethylene (LDPE, LLDPE, VLDPE, ULDPE, MDPE, HDPE, UHMWPE), metallocene PE (m-PE), polypropylene, polyisobutylene, poly-4-methyl-pentene-1, polybutadiene, polyisoprene, polycyclooctene, polyalkylene-carbon monoxide copolymers, and copolymers in the form of random or block structures, such as e.g. polypropylene-polyethylene (EP), EPM or EPDM, ethylene-vinyl acetate (EVA), ethylene-acrylic esters, such as e.g. ethylene-butyl acrylate, ethylene-acrylic acid and its salts (ionomers), and terpolymers, such as e.g. ethylene-acrylic acid-glycidyl (meth)acrylate, graft polymers, such as e.g. polypropylene-graft-maleic anhydride, polypropylene-graft-acrylic acid, polyethylene-graft-acrylic acid, polyethylene-polybutylacrylate-graft-maleic anhydride and blends thereof,
b) polystyrene, polymethylstyrene, poly-alpha-methylstyrene, polyvinylnaphthalene, polyvinylbiphenyl, polyvinyltoluene, styrene-butadiene (SB), styrene-butadiene-styrene (SBS), styrene-ethylene-butylene-styrene (SEBS), styrene-ethylene-propylene-styrene, styrene-isoprene, styrene-isoprene-styrene (SIS), styrene-butadiene-acrylonitrile (ABS), styrene-acrylonitrile (SAN), styrene-acrylonitrile-acrylate (ASA), styrene-ethylene, styrene-maleic anhydride polymers incl. corresponding graft copolymers such as styrene on butadiene, maleic anhydride on SBS or SEBS, and graft copolymers made of methyl methacrylate, styrene-butadiene and ABS (MABS), and hydrogenated polystyrene derivatives,
c) halogen-comprising polymers, such as e.g. polyvinyl chloride (PVC), polychloroprene and polyvinylidene chloride (PVDC), copolymers of vinyl chloride and vinylidene chloride or of vinyl chloride and vinyl acetate, chlorinated polyethylene, polyvinylidene fluoride, epichlorohydrin homo and copolymers thereof,
d) polymers of unsaturated esters, such as e.g. polyacrylates and polymethacrylates like polymethyl methacrylate (PMMA), polybutyl acrylate, polylauryl acrylate, polystearyl acrylate, polyglycidyl acrylate, polyglycidyl methacrylate, polyacrylonitrile, polyacrylamide, copolymers such as e.g. polyacrylonitrile-polyalkyl acrylate,
e) polymers of unsaturated alcohols and derivatives, such as e.g. polyvinyl alcohol, polyvinyl acetate, polyvinyl butyral, polyallyl phthalate, polyallyl melamine,
f) polyacetals such as polyoxymethylene (POM) or copolymers with, for example, butanal, polyphenylene oxides and blends with polystyrene or polyamides,
g) polymers of cyclic ethers, such as e.g. polyethylene glycol, polypropylene glycol, polyethylene oxide, polypropylene oxide, polytetrahydrofuran,
h) polyurethanes made from hydroxy-terminated polyethers or polyesters and aromatic or aliphatic isocyanates, in particular linear polyurethanes (TPU), polyureas,
i) polyamides such as e.g. Polyamide-6, 6.6, 6.10, 4.6, 4.10, 6.12, 10.10, 10.12, 12.12, Polyamide 11, Polyamide 12 and (partially) aromatic polyamides such as e.g. polyphthalamides, e.g. made from terephthalic acid and/or isophthalic acid and aliphatic diamines or from aliphatic dicarboxylic acids such as e.g. adipic acid or sebacic acid and aromatic diamines such as e.g. 1,4- or 1,3-diaminobenzene, blends of different polyamides such as e.g. PA-6 and PA 6.6 or blends of polyamides and polyolefins such as e.g. PA/PP
j) polyimides, polyamide-imides, polyetherimides, polyesterimides, poly(ether)ketones, polysulfones, polyethersulfones, polyarylsulfones, polyphenylene sulfides, polybenzimidazoles, polyhydantoins,
k) polyesters made from aliphatic or aromatic dicarboxylic acids and diols or from hydroxycarboxylic acids, such as e.g. polyethylene terephthalate (PET), polybutylene terephthalate (PBT), polypropylene terephthalate (PTT), polyethylene naphthylate (PEN), poly-1,4-dimethylolcyclohexane terephthalate, polyhydroxybenzoate, polyhydroxynaphthalate, polylactic acid (PLA), polyhydroxybutyrate (PHB), polyhydroxyvalerate (PHV), polyethylene succinate, polytetramethylene succinate, polycaprolactone,
l) polycarbonates, polyester carbonates and blends, such as e.g. PC/ABS, PC/PBT, PC/PET/PBT, PC/PA
m) cellulose derivatives, such as e.g. cellulose nitrate, cellulose acetate, cellulose propionate, cellulose butyrate,
n) epoxy resins, consisting of di- or polyfunctional epoxy compounds in combination with, for example, hardeners based on amines, anhydrides, dicyandiamide, mercaptans, isocyanates or catalytically active hardeners,
o) phenolic resins, such as e.g. phenol-formaldehyde resins, urea-formaldehyde resins, melamine-formaldehyde resins,
p) unsaturated polyester resins from unsaturated dicarboxylic acids and diols with vinyl compounds, alkyd resins, allyl resins
q) silicones, for example based on dimethylsiloxanes, methyl-phenyl-siloxanes or diphenylsiloxanes, for example terminated with vinyl groups
r) and mixtures, combinations or blends of two or more of the aforementioned polymers.

If the polymers specified under a) to r) are copolymers, these can exist in the form of statistical (“random”), block or “tapered” structures. Furthermore, the polymers mentioned can exist in the form of linear, branched, star-shaped or hyperbranched structures.

If the polymers specified under a) to r) are stereoregular polymers, they can exist in the form of isotactic, stereotactic, but also atactic forms or as stereoblock copolymers.

Furthermore, the polymers specified under a) to r) can have both amorphous and (partially) crystalline morphologies.

Optionally, the polyolefins mentioned under a) can also be crosslinked, for example crosslinked polyethylene, which is then referred to as X-PE.

Furthermore, the present compounds can be used to stabilize rubbers and elastomers. This can be natural rubber (NR) or synthetic rubber materials such as NR (Natural Rubber), chloroprene (CR), polybutadiene (BR), styrene-butadiene (SBR), polyisoprene (IR), butyl rubber (IIR), nitrile rubber (NBR), hydrogenated nitrile rubber (HNBR), polyester or polyether urethane rubber, silicone rubber.

Apart from new goods, the plastics can be recycled plastics, for example, from industrial collections such as e.g. production waste or plastics from household or recyclable collections.

Thermoplastic plastics, and in particular plastics that are used in packaging such as e.g. food packaging, in particular polyolefins, polystyrene, polyesters and polyamides, are preferred as plastics. Polypropylene homo- and copolymers are very particularly preferred, and polyethylene in the form of LDPE, LLDPE, HDPE, MDPE, VLDPE and polyethylene terephthalate (PET), homo- and copolymers.

Particular preference is further given to aliphatic polyesters from renewable raw materials, which are substantially produced from aliphatic dicarboxylic acids and aliphatic diols, from hydroxycarboxylic acids or lactones. such as e.g. polylactic acid (PLA), polyglycolic acid (PGA), polyhydroxybutyric acid (PHB), polyhydroxyvaleric acid (PHV), polyethylene succinate (PESu), polybutylene succinate (PBS), polyethylene adipate, poly(butylene succinate-co-adipate) (PBSA) or polycaprolactone (PCL).

For example, the plastic can comprise at least one additional additive selected from the group consisting of primary and/or secondary antioxidants, in particular primary and/or secondary antioxidants selected from the group consisting of phosphites, phosphonites, thiols, phenolic antioxidants, sterically hindered amines, hydroxylamines and mixtures or combinations thereof, UV absorbers, light stabilizers, hydroxylamine-based stabilizers, benzofuranone-based stabilizers, nucleating agents, impact modifiers, plasticizers, lubricants, rheology modifiers, chain extenders, processing aids, pigments, dyes, optical brighteners, antimicrobial agents, antistatic agents, slip agents, antiblocking agents, coupling agents, dispersants, compatibilizers, oxygen scavengers, acid scavengers, co-stabilizers, marking agents and anti-fogging agents, and/or can be added to the plastic during the use.

In the event that an additive or a plurality of additives are contained in the plastic, said additives are preferably contained or added during use in a total amount of 0.01 to 9.99% by weight, preferably 0.01 to 4.98% by weight, particularly preferably 0.02 to 2.00% by weight, based on the total of the at least one compound according to Formula I, the organic material and the at least one additive.

The present invention also relates to an organic material, in particular a plastics composition, comprising at least one compound of general Formula I or a mixture of a plurality of compounds according to general Formula I as a stabilizer

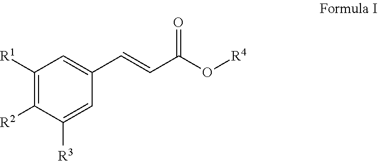

where R¹, R², Wand R⁴ are as defined in any one of claims 1 and 5.

All the preferred versions as formulated above regarding the compound according to Formula I also apply without restriction to the organic material according to the invention.

The organic material preferably has the following composition:

0.01 to 10.00% by weight, preferably from 0.02 to 5.00% by weight, particularly preferably from 0.10 to 2.00% by weight, of a compound according to general Formula I, or, in the case of a mixture of a plurality of compounds according to general Formula I, the entirety of all compounds according to general Formula I
99.99 to 90.00% by weight, preferably 99.89 to 95.00% by weight, particularly preferably 99.90 to 98.00% by weight of at least one organic material, preferably selected from the group consisting of plastics, coatings, lubricants, hydraulic oils, engine oils, turbine oils, gear oils, metalworking fluids, chemicals or monomers, and
0 to 9.99% by weight, preferably 0 to 4.98% by weight, particularly preferably 0.02 to 2.00% by weight of at least one additive,
the components adding up to 100% by weight.

According to a further preferred embodiment, the at least one additive is selected from the group consisting of primary and/or secondary antioxidants, in particular primary and/or secondary antioxidants selected from the group consisting of phosphites, phosphonites, thiols, phenolic antioxidants, sterically hindered amines, hydroxylamines and mixtures or combinations thereof, UV absorbers, light stabilizers, hydroxylamine-based stabilizers, benzofuranone-based stabilizers, nucleating agents, impact modifiers, plasticizers, lubricants, rheology modifiers, chain extenders, processing aids, pigments, dyes, optical brighteners, antimicrobial agents, antistatic agents, slip agents, antiblocking agents, coupling agents, dispersants, compatibilizers, oxygen scavengers, acid scavengers, co-stabilizers, marking agents and anti-fogging agents;

is selected in particular from the group consisting of a secondary antioxidant selected from the group consisting of phosphites, phosphonites and thiols, at least one co-stabilizer selected from the group consisting of polyols, acid scavengers and sterically hindered amines.

Suitable primary antioxidants (A) are phenolic antioxidants, amines and lactones.

Suitable synthetic phenolic antioxidants are, for example:

Alkylated monophenols, such as 2,6-di-tert-butyl-4-methylphenol, 2-tert-butyl-4,6-dimethylphenol, 2,6-di-tert-butyl-4-ethylphenol, 2,6-di-tert-butyl-4-n-butylphenol, 2,6-di-tert-butyl-4-isobutylphenol, 2,6-dicyclopentyl-4-methylphenol, 2-(α-methylcyclohexyl)-4,6-dimethylphenol, 2,6-dioctadecyl-4-methylphenol, 2,4,6-tricyclohexylphenol, 2,6-di-tert-butyl-4-methoxymethylphenol, linear or branched nonylphenols such as 2,6-dinonyl-4-methylphenol, 2,4-dimethyl-6-(1′-methylundec-1′-yl)phenol, 2,4-dimethyl-6-(1′-methylheptadec-1′-yl)phenol, 2,4-dimethyl-6-(1′-methyltridec-1′-yl)phenol and mixtures thereof;
alkylthiomethyl phenols, such as e.g. 2,4-dioctylthiomethyl-6-tert-butylphenol, 2,4-dioctylthiomethyl-6-methylphenol, 2,4-dioctylthiomethyl-6-ethylphenol, 2,6-didodecylthiomethyl-4-nonylphenol;
hydroquinones and alkylated hydroquinones such as e.g. 2,6-di-tert-butyl-4-methoxyphenol, 2,5-di-tert-butylhydroquinone, 2,5-di-tert-amylhydroquinone, 2,6-diphenyl-4-octadecyloxyphenol, 2,6-di-tert-butylhydroquinone, 2,5-di-tert-butyl-4-hydroxyanisole, 3,5-di-tert-butyl-4-hydroxyanisole, 3,5-di-tert-butyl-4-hydroxyphenyl stearate, bis(3,5-di-tert-butyl-4-hydroxyphenyl) adipate;
tocopherols, such as e.g. α-, β-, γ-, δ-tocopherol and mixtures thereof (vitamin E);
hydroxylated thiodiphenyl ethers, such as 2,2′-thiobis(6-tert-butyl-4-methylphenol), 2,2′-thiobis(4-octylphenol), 4,4′-thiobis(6-tert-butyl-3-methylphenol), 4,4′-thiobis(6-tert-butyl-2-methylphenol), 4,4′-thiobis(3,6-di-sec-amylphenol), 4,4′-bis(2,6-dimethyl-4-hydroxyphenyl)disulfide;
alkylidene bisphenols, such as 2,2′methylenebis(6-tert-butyl-4-methylphenol), 2,2′-methylenebis(6-tert-butyl-4-ethylphenol), 2,2′-methylenebis[4-methyl-6-(α-methylcyclohexyl)phenol], 2,2′-methylenebis(4-methyl-6-cyclhexylphenol), 2,2′-methylenebis(6-nonyl-4-methylphenol), 2,2′-methylenebis(4,6-di-tert-butylphenol), 2,2′-ethylidenebis(4,6-di-tert-butylphenol), 2,2′-ethylidenebis(6-tert-butyl-4-isobutylphenol), 2,2′-methylenebis[6-(α-methylbenzyl)-4-nonylphenol], 2,2′-methylenebis[6-(α,α-dimethylbenzyl)-4-nonylphenol], 4,4′-methylenebis(2,6-di-tert-butylphenol, 4,4′-methylenebis(6-tert-butyl-2-methylphenol), 1,1-bis(5-tert-butyl-4-hydroxy methylphenyl)butane, 2,6-bis(3-tert-butyl-5-methyl-2-hydroxybenzyl)-4-methylphenol, 1,1,3-tris(5-tert-butyl-4-hydroxy-2-methylphenyl)butane, 1,1-bis(5-tert-butyl-4-hydroxy methylphenyl)-3-n-dodecylmercaptobutane, ethylene glycol-bis[3,3-bis(3′-tert-butyl-4′-hydroxyphenyl)butyrate], bis(3-tert-butyl-4-hydroxy-5-methylphenyl)dicyclopentadiene, bis[2-(3′-tert-butyl-2′-hydroxy-5′-methylbenzyl)-6-tert-butyl-4-methylphenyl]terephthalate, 1,1-bis-(3,5-dimethyl-2-hydroxyphenyl)butane, 2,2-bis(3,5-di-tert-butyl-4-hydroxyphenyl)propane, 2,2-bis-(5-tert-butyl-4-hydroxy-2-methylphenyl)-4-n-dodecylmercaptobutane, 1,1,5,5-tetra(5-tert-butyl-4-hydroxy-2-methylphenyl)pentane;
O-, N- and S-benzyl compounds, such as 3,5,3′,5′-tetra-tert-butyl-4,4′-dihydroxydibenzylether, octadecyl-4-hydroxy-3,5-dimethylbenzylmercaptoacetate, tridecyl-4-hydroxy-3,5-di-tert-butylbenzylmercaptoacetate, tris(3,5-di-tert-butyl-4-hydroxybenzyl)amine, bis(4-tert-butyl-3-hydroxy-2,6-dimethylbenzyl)dithioterephthalate, bis(3,5-di-tert-butyl-4-hydroxybenzyl)sulfide, isooctyl-3,5-di-tert-butyl-4-hydroxybenzylmercaptoacetate;
hydroxybenzylated malonates, such as dioctadecyl-2,2-bis(3,5-di-tert-butyl-2-hydroxybenzyl)malonate, dioctadecyl-2-(3-tert-butyl-4-hydroxy-5-methylbenzyl)malonate, didodecylmercaptoethyl-2,2-bis(3,5-di-tert-butyl-4-hydroxybenzyl)malonate, bis[4-(1,1,3,3-tetramethylbutyl)phenyl]-2,2-bis(3,5-di-tert-butyl-4-hydroxybenzyl)malonate;
aromatic hydroxybenzyl compounds, such as 1,3,5-tris(3,5-di-tert-butyl-4-hydroxybenzyl)-2,4,6-trimethylbenzene, 1,4-bis(3,5-di-tert-butyl-4-hydroxybenzyl)-2,3,5,6-tetramethylbenzene, 2,4,6-tris(3,5-di-tert-butyl-4-hydroxybenzyl)phenol;
triazine compounds, such as 2,4-bis(octylmercapto)-6-(3,5-di-tert-butyl-4-hydroxyanilino)-1,3,5-triazine, 2-octylmercapto-4,6-bis(3,5-di-tert-butyl-4-hydroxyanilino)-1,3,5-triazine, 2-octylmercapto-4,6-bis(3,5-di-tert-butyl-4-hydroxyphenoxy)-1,3,5-triazine, 2,4,6-tris(3,5-di-tert-butyl-4-hydroxyphenoxy)-1,2,3-triazine, 1,3,5-tris(3,5-di-tert-butyl hydroxybenzyl)isocyanurate, 1,3,5-tris(4-tert-butyl-3-hydroxy-2,6-dimethylbenzyl)isocyanurate, 2,4,6-tris(3,5-di-tert-butyl-4-hydroxphenylethyl)-1,3,5-triazine, 1,3,5-tris(3,5-di-tert-butyl-4-hydroxyphenylpropionyl)hexahydro-1,3,5-triazine, 1,3,5-tris(3,5-dicyclohexyl-4-hydroxybenzyl)isocyanurate;
benzyl phosphonates, such as dimethyl-2,5-di-tert-butyl-4-hydroxybenzylphosphonate, diethyl-3,5-di-tert-butyl-4-hydroxybenzylphosphonate, dioctadecyl-3,5-di-tert-butyl hydroxybenzylphosphonate, dioctadecyl-5-tert-butyl-4-hydroxy-3-methylbenzylphosphonate, the calcium salt of the monoethylester of 3,5-di-tert-butyl-4-hydroxybenzylphosphonic acid;
acylaminophenols, such as 4-hydroxylauranilide, 4-hydroxystearanilide, octyl-N-(3,5-di-tert-butyl-4-hydroxyphenyl)carbamate;
esters of β-(3,5-di-tert-butyl-4-hydroxyphenyl)propionic acid with monohydric or polyhydric alcohols, for example methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane;
esters of β-(5-tert-butyl-4-hydroxy-3-methylphenyl)propionic acid with monohydric or polyhydric alcohols, for example methanol, ethanol, n-octanol, i-octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane, 3,9-bis[2-{3-(3-tert-butyl-4-hydroxy-5-methylphenyl)propionyloxy}-1,1-dimethylethyl]-2,4,8,10-tetraoxaspiro[5.5]undecane;
esters of β-(3,5-dicyclohexyl-4-hydroxyphenyl)propionic acid with monohydric or polyhydric alcohols, for example methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane;
esters of (3,5-di-tert-butyl-4-hydroxyphenyl)acetic acid with monohydric or polyhydric alcohols, for example methanol, ethanol, octanol, octadecanol, 1,6-hexanediol, 1,9-nonanediol, ethylene glycol, 1,2-propanediol, neopentyl glycol, thiodiethylene glycol, diethylene glycol, triethylene glycol, pentaerythritol, tris(hydroxyethyl)isocyanurate, N,N′-bis(hydroxyethyl)oxamide, 3-thiaundecanol, 3-thiapentadecanol, trimethylhexanediol, trimethylolpropane, 4-hydroxymethyl-1-phospha-2,6,7-trioxabicyclo[2.2.2]octane;
amides of β-(3,5-di-tert-butyl-4-hydroxyphenyl)propionic acid, such as N,N′-bis(3,5-di-tert-butyl-4-hydroxyphenylpropionyl)hexamethylene diamide, N,N′-bis(3,5-di-tert-butyl-4-hydroxyphenylpropionyl)hexamethylene diamide, N,N′-bis(3,5-di-tert-butyl-4-hydroxyphenylpropionyl)hexamethylene diamide, N,N′-bis(3,5-di-tert-butyl-4-hydroxyphenylpropionyl)hydrazide, N,N′-bis[2-(3-[3,5-di-tert-butyl-4-hydroxyphenyl]propionyloxy)ethyl]oxamide (Naugard® XL-1, marketed by Uniroyal);
ascorbic acid (vitamin C).

Particularly preferred phenolic antioxidants are the following structures:

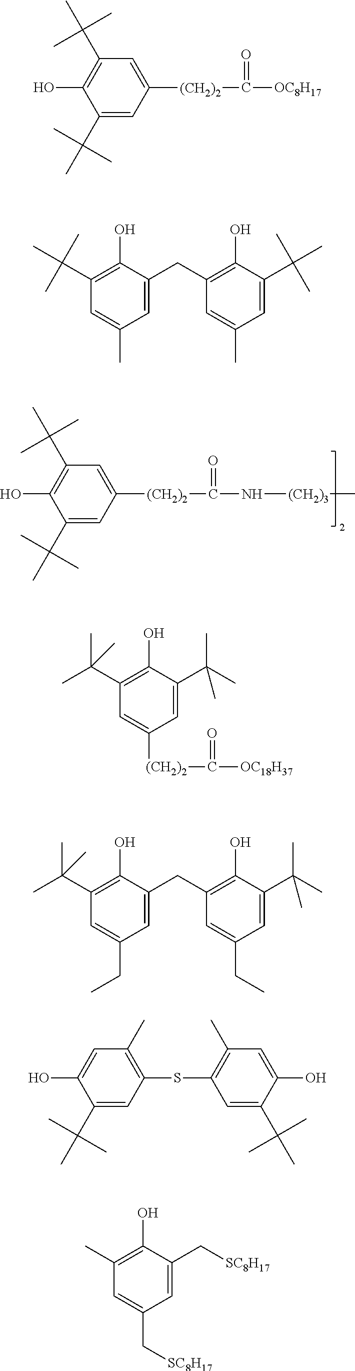

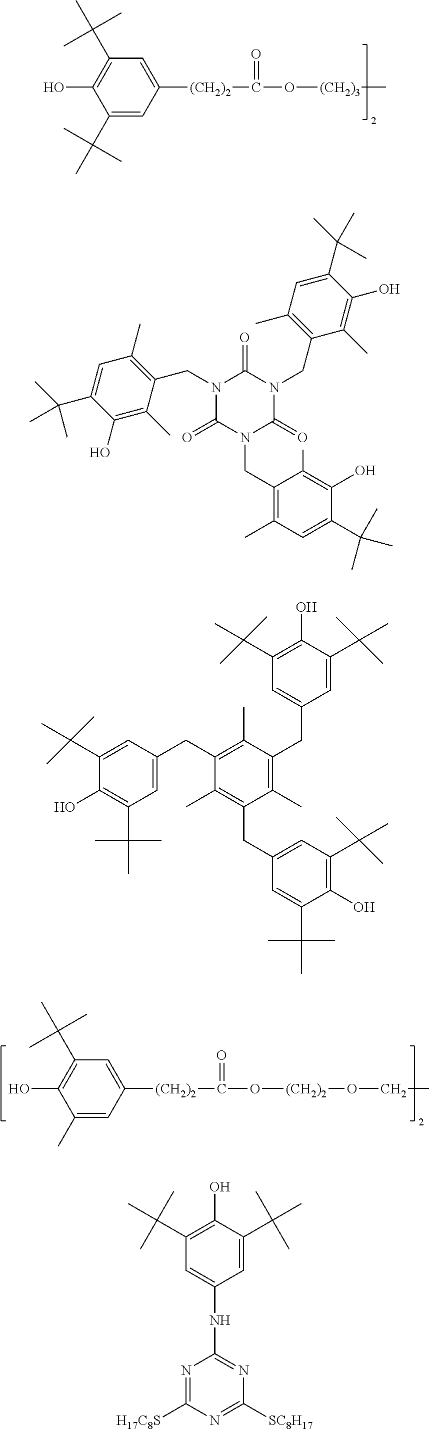

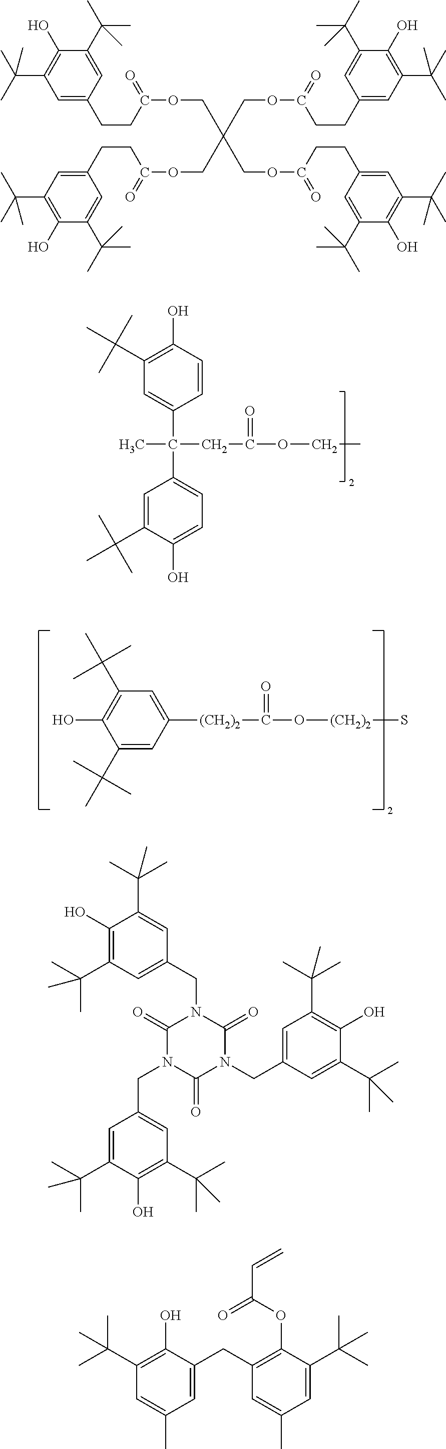

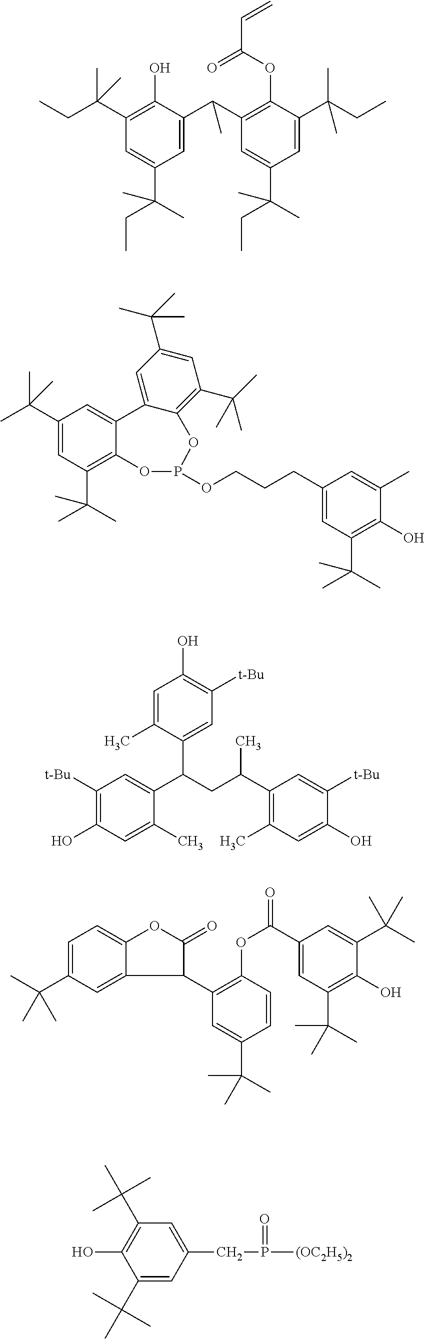

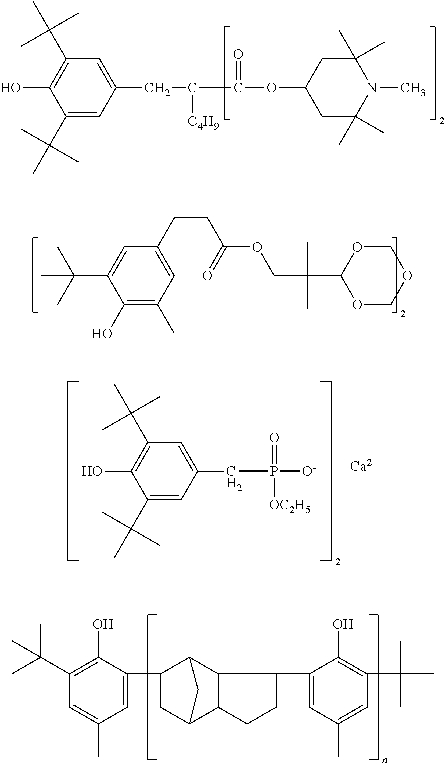

with n=2 to 10

Very particularly preferred phenolic antioxidants are octadecyl-3-(3,5-di-tert-butyl-4-hydroxyphenyl)propionate and pentaerythritol tetrakis(3-(3,5-di-tert-butyl-4-hydroxyphenyl)propionate).

Further particularly preferred phenolic antioxidants are based on renewable raw materials such as e.g. tocopherols (vitamin E), tocotrienols, tocomomonoenols, carotenoids, hydroxytyrosol, flavonols such as e.g. chrysin, quercitin, hesperidin, neohesperidin, naringin, morin, kaempferol, fisetin, anthocyanins such as e.g. delphinidin and malvidin, curcumin, carnosolic acid, carnosol, rosmarinic acid and resveratrol.

Suitable aminic antioxidants are, for example:

N,N′-di-isopropyl-p-phenylene diamine, N,N′-di-sec-butyl-p-phenylene diamine, N,N′-bis(1,4-dimethylpentyl)-p-phenylene diamine, N,N′-bis(1-ethyl-3-methylpentyl)-p-phenylene diamine, N,N′-bis(1-methylheptyl)-p-phenylene diamine, N,N′-dicyclohexyl-p-phenylene diamine, N,N′-diphenyl-p-phenylene diamine, N,N′-bis(2-naphthyl)-p-phenylene diamine, N-isopropyl-N′-phenyl-p-phenylene diamine, N-(1,3-dimethylbutyl)-N′-phenyl-p-phenylene diamine, N-(1-methylheptyl)-N′-phenyl-p-phenylene diamine, N-cyclohexyl-N′-phenyl-p-phenylene diamine, 4-(p-toluene sulfamoyl)diphenylamine, N,N′-dimethyl-N,N′-di-sec-butyl-p-phenylene diamine, diphenylamine, N-allyldiphenylamine, 4-isopropoxydiphenylamine, N-phenyl-1-naphthylamine, N-(4-tert-octylphenyl)-1-naphthylamine, N-phenyl-2-naphthylamine, octylated diphenylamine, for example p,p′-di-tert-octyldiphenylamine, 4-n-butylaminophenol, 4-butyrylaminophenol, 4-nonanoylaminophenol, 4-dodecanoylaminophenol, 4-octadecanoylamino-phenol, bis(4-methoxyphenyl)amine, 2,6-di-tert-butyl-4-dimethylaminomethyl-phenol, 2,4′-diaminodiphenylmethane, 4,4′-diaminodiphenylmethane, N,N,N′,N′-tetra-methyl-4,4′-diaminodiphenylmethane, 1,2-bis[(2-methyl-phenyl)amino]ethane, 1,2-bis(phenylamino)propane, (o-tolyl)biguanide, bis[4-(1′,3′-dimethylbutyl)phenyl]amine, tert-octylated N-phenyl-1-naphthylamine, a mixture of mono- and dialkylated tert-butyl/tert-octyldiphenylamines, a mixture of mono- and dialkylated nonyldiphenylamines, a mixture of mono- and dialkylated dodecyldiphenylamines, a mixture of mono- and dialkylated isopropyl/isohexyl-diphenylamines, a mixture of mono- and dialkylated tert-butyldiphenylamines, 2,3-dihydro-3,3-dimethyl-4H-1,4-benzothiazine, phenothiazine, a mixture of mono- and dialkylated tert-butyl/tert-octylphenothiazines, a mixture of mono- and dialkylated tert-octylphenothiazinene, N-allylphenothiazine, N,N,N′,N′-tetraphenyl-1,4-diaminobut-2-ene and mixtures or combinations hereof.

Preferred aminic antioxidants are: N,N′-di-isopropyl-p-phenylenediamine, N,N′-di-sec-butyl-p-phenylenediamine, N,N′-bis(1,4-dimethylpentyl)-p-phenylenediamine, N,N′-bis(1-ethyl-3-methylpentyl)-p-phenylenediamine, N,N′-bis(1-methylheptyl)-p-phenylenediamine, N,N′-dicyclohexyl-p-phenylenediamine, N,N′-diphenyl-p-phenylenediamine, N,N′-bis(2-naphthyl)-p-phenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenylenediamine

Particularly preferred aminic antioxidants are the structures:

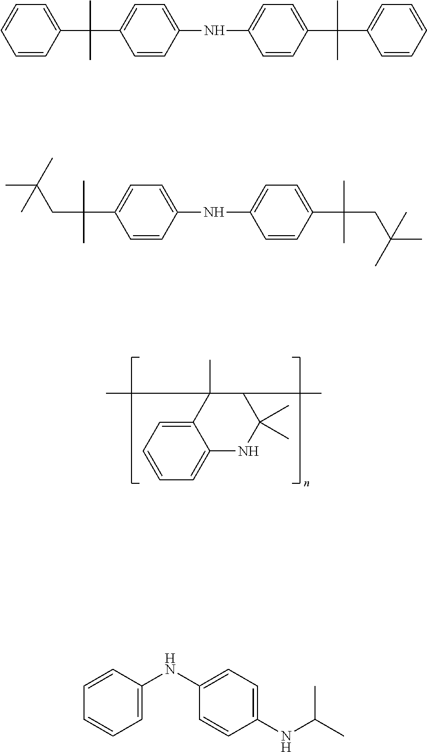

with n=3-100

Further preferred aminic antioxidants are hydroxylamines or N-oxides (nitrones), such as e.g. N,N-dialkylhydroxylamines, N,N-dibenzylhydroxylamine, N,N-dilaurylhydroxylamine, N,N-distearylhydroxylamine, N-benzyl-α-phenylnitrone, N-octadecyl-α-hexadecylnitrone, and Genox EP (SI Group) according to the formula:

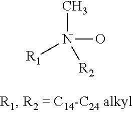

Suitable lactones are benzofuranones and indolinones such as 3-(4-(2-acetoxyethoxy)-phenyl]-5,7-di-tert-butyl-benzofuran-2-one, 5,7-di-tert-butyl-3-[4-(2-stearoyloxyethoxy)phenyl]benzofuran-2-one, 3,3′-bis[5,7-di-tert-butyl-3-(4-(2-hydroxyethoxy] phenyl)benzofuran-2-one), 5,7-di-tert-butyl-3-(4-ethoxyphenyl)benzofuran-2-one, 3-(4-acetoxy-3,5-dimethylphenyl)-5,7-di-tert-butyl-benzofuran-2-one, 3-(3,5-dimethyl-4-pivaloyloxyphenyl)-5,7-di-tert-butyl-benzofuran-2-one, 3-(3,4-dimethylphenyl)-5,7-di-tert-butyl-benzofuran-2-one, 3-(2,3-dimethylphenyl)-5,7-di-tert-butyl-benzofuran-2-one and lactones that also comprise phosphite groups such as

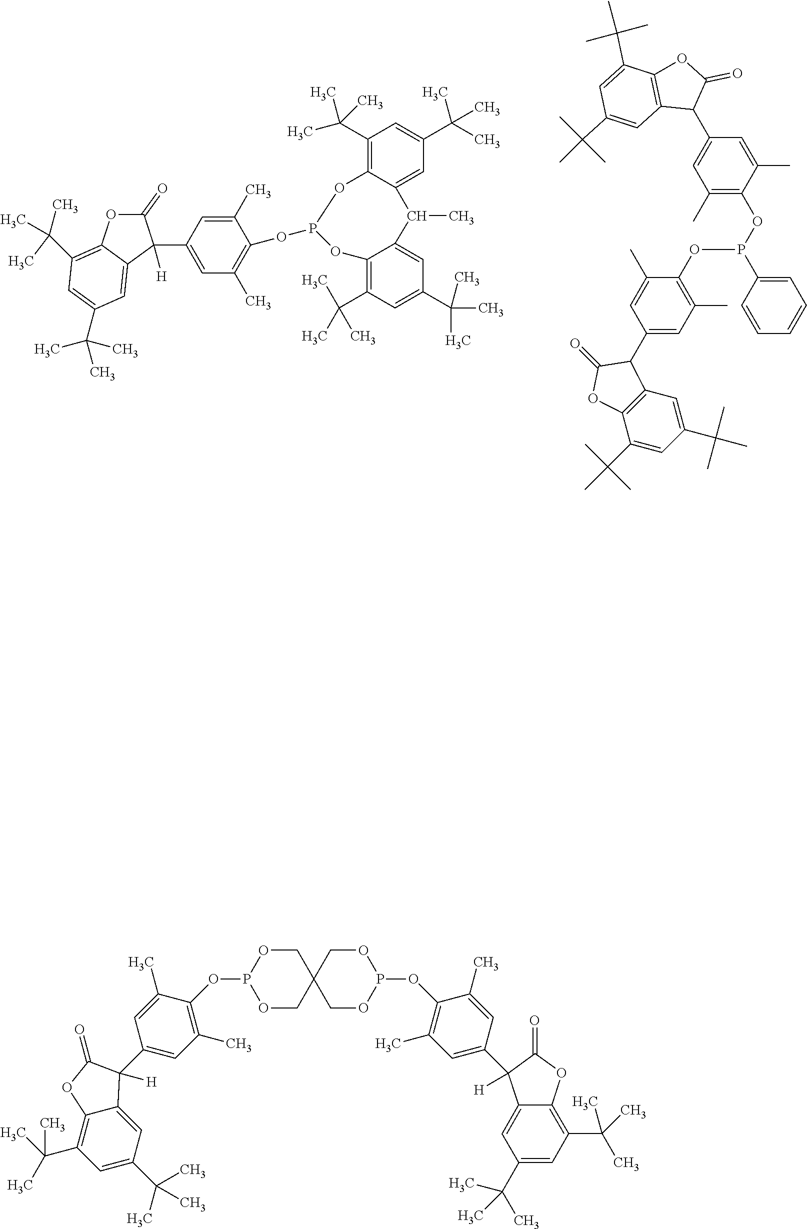 [SMILES: Cc1cc(C2C(=O)Oc3c2cc(C(C)(C)C)cc3C(C)(C)C)cc(C)c1OP1Oc2c(cc(C(C)(C)C)cc2C(C)(C)C)C(C)c2cc(C(C)(C)C)cc(C(C)(C)C)c2O1 ; Cc1cc(C2C(=O)Oc3c2cc(C(C)(C)C)cc3C(C)(C)C)cc(C)c1OP(Oc1c(C)cc(C2C(=O)Oc3c2cc(C(C)(C)C)cc3C(C)(C)C)cc1C)c1ccccc1 ; Cc1cc(C2C(=O)Oc3c2cc(C(C)(C)C)cc3C(C)(C)C)cc(C)c1OP1OCC2(CO1)COP(Oc1c(C)cc(C3C(=O)Oc4c3cc(C(C)(C)C)cc4C(C)(C)C)cc1C)OC2]

A further suitable group of antioxidants are isoindolol[2,1-A]quinazolines such as e.g.

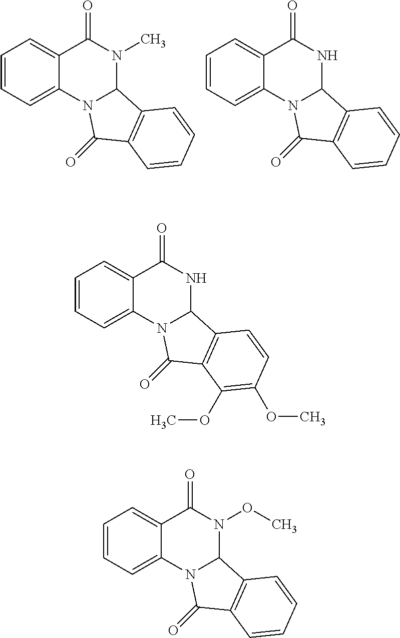

Suitable secondary antioxidants are in particular phosphites or phosphonites such as e.g. triphenylphosphite, diphenylalkylphosphite, phenyldialkylphosphite, tri(nonylphenyl)phosphite, trilaurylphosphite, trioctadecylphosphite, distearylpentaerythritoldiphosphite, tris-(2,4-di-tert-butylphenyl)phosphite, diisodecylpentaerythritoldiphosphite, bis(2,4-di-tert-butylphenyl)pentaerythritoldiphosphite, bis(2,4-di-cumylphenyl)pentaerythritoldiphosphite, bis(2,6-di-tert-butyl-4-methylphenyl)pentaerythritoldiphosphite, diisodecyloxypentaerythritoldiphosphite, bis(2,4-di-tert-butyl-6-methylphenyl)pentaerythritoldiphosphite, bis(2,4,6-tris(tert-butylphenyl)pentaerythritoldiphosphite, tristearylsorbitoltriphosphite, tetrakis(2,4-di-tert-butylphenyl)-4,4′-biphenylene diphosphonite, 6-isooctyloxy-2,4,8,10-tetra-tert-butyl-12H-dibenz[d,g]-1,3,2-dioxaphosphocine, bis(2,4-di-tert-butyl-6-methylphenyl)methylphosphite, bis(2,4-di-tert-butyl-6-methylphenyl)ethylphosphite, 6-fluoro-2,4,8,10-tetra-tert-butyl-12-methyl-dibenz[d,g]-1,3,2-dioxaphosphocine, 2,2′2″-nitrilo[triethyltris(3,3″,5,5′-tetra-tert-butyl-1,1′-biphenyl-2,2′-diyl)phosphite], 2-ethylhexyl(3,3′,5,5′-tetra-tert-butyl-1,1′-biphenyl-2,2′-diyl))phosphite, 5-butyl-5-ethyl-2-(2,4,6-tri-tert-butylphenoxy)-1,3,2-dioxaphosphirane.

Particularly preferred phosphites/phosphonites are:

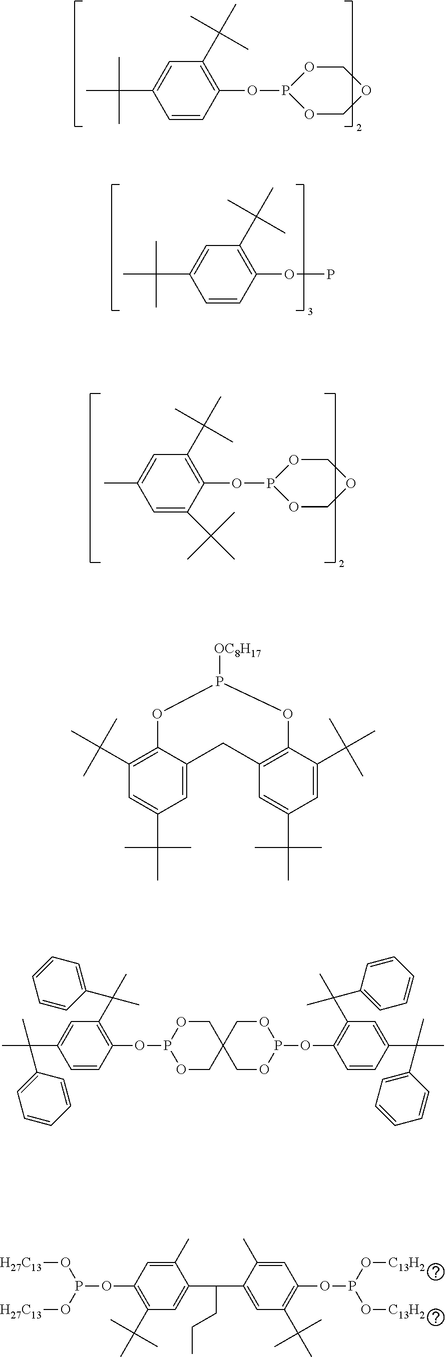

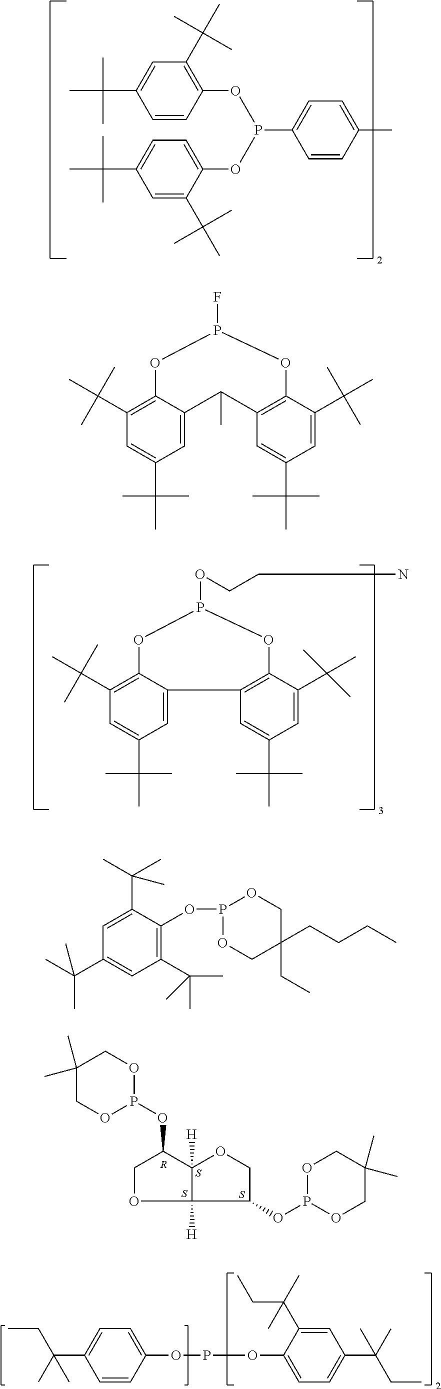

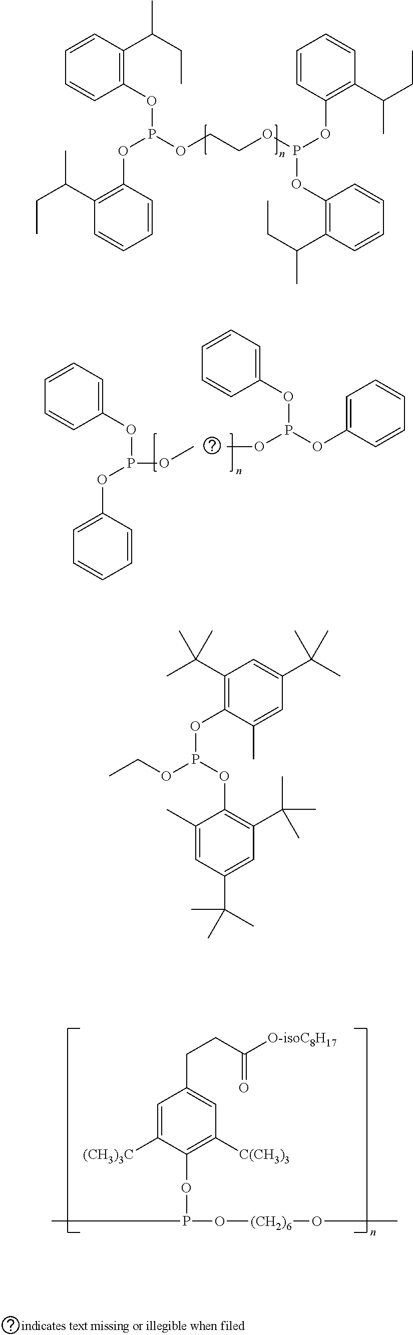

with n=3-100

A preferred phosphonite is:

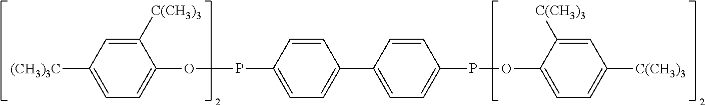

The phosphite tris-(2,4-di-tert-butylphenyl)phosphite is very particularly preferably used as the secondary antioxidant.

Suitable secondary antioxidants are furthermore organosulfur compounds such as sulfides and disulfides, e.g. distearylthiodipropionate, dilaurylthiodipropionate; ditridecyldithiopropionate, ditetradecylthiodipropionate, 3-(dodecylthio)-,1,1′-[2,2-bis[[3-(dodecylthio)-1-oxopropoxy]methyl]-1,3-propandiyl] propanoic acid ester. The following structures are preferred:

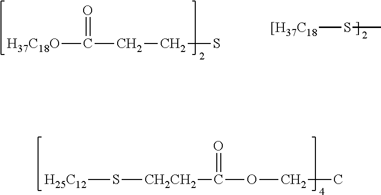

Suitable acid scavengers (“antacids”) are salts of monovalent, bivalent, trivalent, or quadrivalent metals, preferably alkali metals, alkaline earth metals, aluminum or zinc, in particular formed with fatty acids such as calcium stearate, magnesium stearate, zinc stearate, aluminum stearate, calcium laurate, calcium behenate, calcium lactate, calcium stearolyl-2-lactate. Further classes of suitable acid scavengers are hydrolactites, in particular synthetic hydrolactites on the basis of aluminum, magnesium and zinc, hydrocalumites, zeolites, alkaline earth oxides, in particular calcium oxide and magnesium oxide and zinc oxide, alkaline earth carbonates, in particular calcium carbonate, magnesium carbonate, dolomite, and hydroxides, in particular brucite (magnesium hydroxide),

Suitable costabilizers are furthermore polyols, in particular alditols or cyclitols. Polyols are, for example, pentaerythritol, dipentaerythritol, tripentaerythritol, short-chain polyether polyols or polyester polyols, and hyperbranched polymers/oligomers or dendrimers having alcohol groups, for example

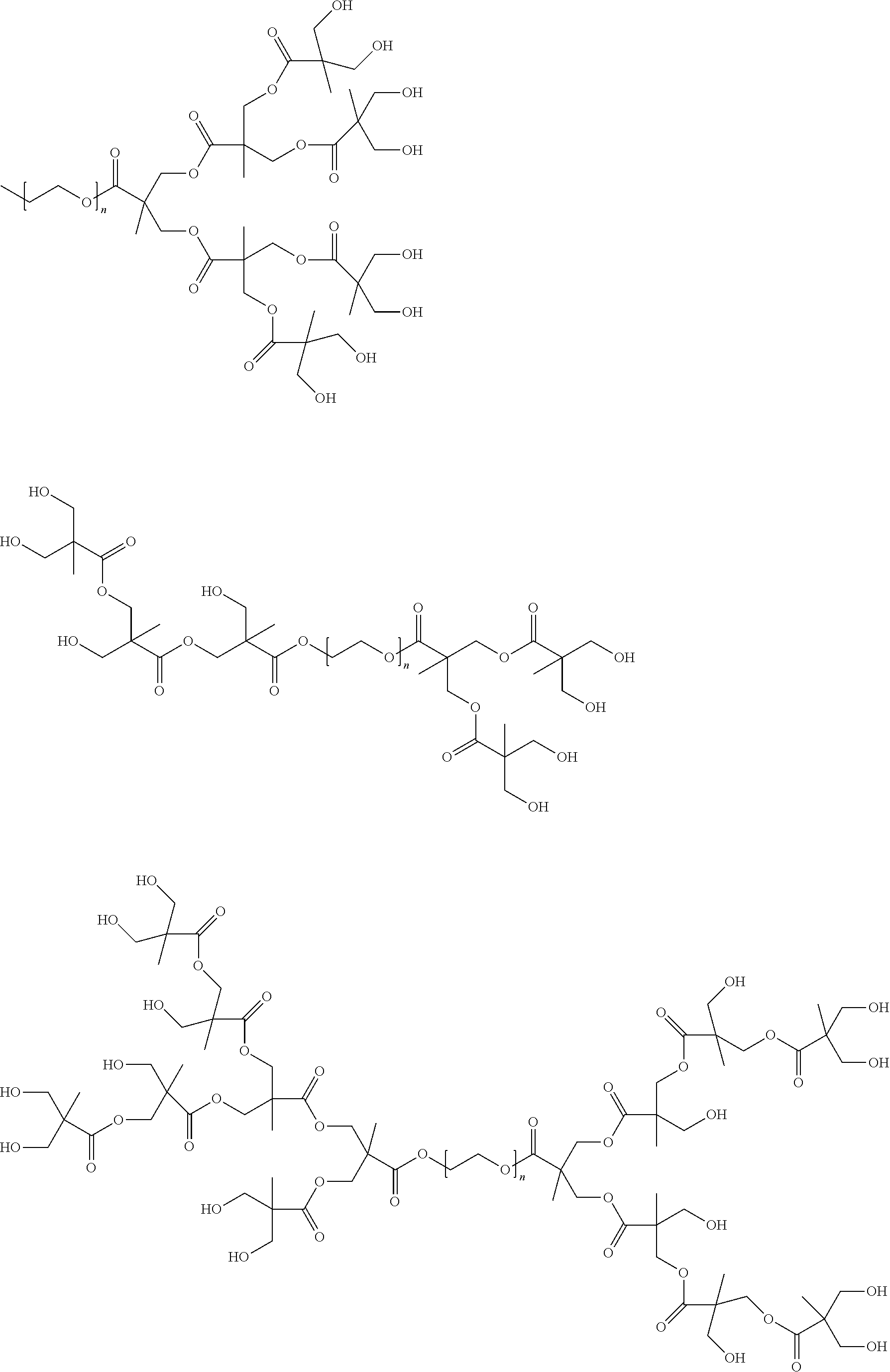

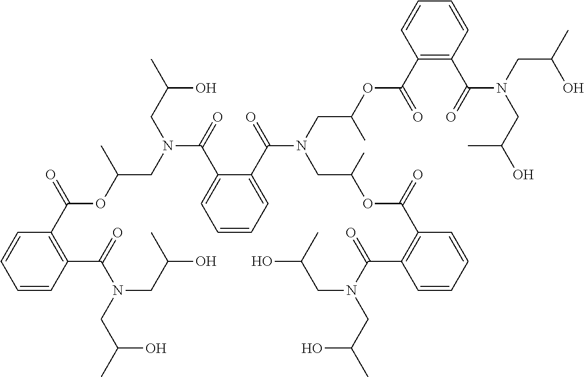 [SMILES: CC(O)CN(CC(C)O)C(=O)c1ccccc1C(=O)OC(C)CN(CC(C)O)C(=O)c1ccccc1C(=O)N(CC(C)OC(=O)c1ccccc1C(=O)N(CC(C)O)CC(C)O)CC(C)OC(=O)c1ccccc1C(=O)N(CC(C)O)CC(C)O]

The at least one alditol is preferably selected from the group consisting of threitol, erythritol, galactitol, mannitol, ribitol, sorbitol, xylitol, arabitol, isomalt, lactitol, maltitol, altritol, iditol, maltotritol and hydrogenated oligo- and polysaccharides having polyol end groups and mixtures thereof. The at least one preferred alditol is particularly preferably selected from the group consisting of erythritol, mannitol, isomaltol, maltitol, and mixtures thereof.

Examples of further suitable sugar alcohols are heptitols and octitols: meso-glycero-allo-heptitol, D-glycero-D-altro-heptitol, D-glycero-D-manno-heptitol, meso-glycero-gulo-heptitol, D-glycero-D-galacto-heptitol (perseitol), D-glycero-D-gluco-heptitol, L-glycero-D-gluco heptitol, D-erythro-L-galacto-octitol, D-threo-L-galacto-octitol.

In particular, the at least one cyclitol may be selected from the group consisting of inositol (myo, scyllo-, D-chiro-, L-chiro-, muco-, neo-, allo-, epi- and cis-inositol), 1,2,3,4-tetrahydroxycyclohexane, 1,2,3,4,5-pentahydroxycyclohexane, quercitol, viscumitol, bornesitol, conduritol, ononitol, pinitol, pinpollitol, quebrachitol, ciceritol, quinic acid, shikimic acid and valienol; in this case myo-inositol is preferred.

Suitable light stabilizers are, for example, compounds based on 2-(2′-hydroxyphenyl)benzotriazoles, 2-hydroxybenzophenones, esters of benzoic acids, acrylates, oxamides and 2-(2-hydroxyphenyl)-1,3,5-triazines.

Suitable 2-(2″-hydroxyphenyl)benzotriazoles are, for example, 2-(2′-hydroxy-5′methylphenyl)benzotriazole, 2-(3′,5′-di-tert-butyl-2′-hydroxyphenyl)benzotriazole, 2-(5′-tert-butyl-2′-hydroxy-phenyl)benzotriazole, 2-(2′-hydroxy-5′-(1,1,3,3-tetramethylbutyl)phenyl)benzotriazole, 2-(3′,5′-di-tert-butyl-2′-hydroxyphenyl)-5-chlorobenzotriazole, 2-(3′-tert-butyl-2′-hydroxy-5′-methylphenyl-5-chlorobenzotriazole, 2-(3′-sec-butyl-5′-tert-butyl-2′-hydroxy-phenyl)benzotriazole, 2-(2′-hydroxy-4′-octyloxyphenyl)benzotriazole, 2-(3′,5′-di-tert-amyl-2′-hydroxyphenyl)benzotriazole, 2-(3′,5′-bis(α,α-dimethylbenzyl)-2′-hydroxyphenyl)benzotriazole, 2-(3′-tert-butyl-2′-hydroxy-5′-(2-octyloxycarbonylethyl)phenyl)-5-chlorobenzotriazole, 2-(3′-tert-butyl-5′-[2-(2-ethylhexyloxy)carbonylethyl]-2′-hydroxyphenyl)-5-chlorobenzotriazole, 2-(3′-tert-butyl-2′-hydroxy-5′-(2-methoxycarbonylethyl)phenyl)-5-chlorobenzotriazole, 2-(3′-tert-butyl-2′-hydroxy-5′-(2-methoxycarbonylethyl)phenyl)benzotriazole, 2-(3′-tert-butyl-2′-hydroxy-5′-(2-octyloxycarbonylethyl)phenyl)benzotriazole, 2-(3′-tert-butyl-5′-[2-(2-ethylhexyloxy)carbonylethyl]-2′-hydroxyphenyl)benzotriazole, 2-(3′-dodecyl-2′-hydroxy-5′-methylphenyl)benzotriazole, 2-(3′-tert-butyl-2′-hydroxy-5′-(2-isooctyloxycarbonylethyl)phenylbenzotriazole, 2,2′-methylenebis[4-(1,1,3,3-tetramethylbutyl)-6-benzotriazole-2-ylphenol]; the product of the transesterification of 2-[3′-tert-butyl-5′-(2-methoxycarbonylethyl)-2′-hydroxyphenyl]-2H-benzotriazole with polyethyleneglycol 300; [R—CH₂CH₂—COO—CH₂CH₂]—₂, where R=3′-tert-butyl-4′-hydroxy-5′-2H-benzotriazole-2-ylphenyl, 2-[2′-hydroxy-3′-(α,α-dimethylbenzyl)-5′-(1,1,3,3-tetramethylbutyl)phenyl]benzotriazole, 2-[2′-hydroxy-3′-(1,1,3,3-tetramethylbutyl)-5′-(α,α-dimethylbenzyl)phenyl]benzotriazole.

Suitable 2-hydroxybenzophenones are, for example, 4-hydroxy-, 4-methoxy-, 4-octyloxy-, 4-decyloxy-4-dodecyloxy, 4-benzyloxy, 4,2′,4′-trihydroxy- and 2′-hydroxy-4,4′-dimethyoxy derivatives of the 2-hydroxy benzophenones.

Suitable acrylates are, for example, ethyl-α-cyano-β,β-diphenylacrylate, isooctyl-α-cyano-β,β-diphenylacrylate, methyl-α-carbomethoxycinnamate, methyl-α-cyano-β-methyl-p-methoxycinnamate, butyl-α-cyano-β-methyl-p-methoxycinnamate, methyl-α-carbomethoxy-p-methoxycinnamate and N-β-carbomethoxy-β-cyanovinyl)-2-methylindoline.

Suitable esters of benzoic acids are, for example, 4-tert-butylphenylsalicylate, phenylsalicylate, octylphenylsalicylate, dibenzoylresorcinol, bis(4-tert-butylbenzoyl)resorcinol, benzoylresorcinol, 2,4-di-tert-butylphenyl-3,5-di-tert-butyl-4-hydroxybenzoate, hexadecyl-3,5-di-tert-butyl-4-hydroxybenzoate, octadecyl-3,5-di-tert-butyl-4-hydroxybenzoate, 2-methyl-4,6-di-tert-butylphenyl-3,5-di-tert-butyl-4-hydroxybenzoate.

Suitable oxamides are, for example, 4,4′-dioctyloxyoxanilide, 2,2′-diethoxyoxanilide, 2,2′-dioctyloxy-5,5′-di-tert-butoxanilide, 2,2′-didodecyloxy-5,5′-di-tert-butoxanilide, 2-ethoxy-2′-ethyloxanilide, N,N′-bis(3-dimethylaminopropyl)oxamide, 2-ethoxy-5-tert-butyl-2′-ethoxanilide and its mixtures with 2-ethoxy-2′-ethyl-5,4′-di-tert-butoxanilide, mixtures of n- and p-methoxy-disubstituted oxanilides and mixtures of o- and p-ethoxy-disubstituted oxanilides.

Suitable 2-(2-hydroxyphenyl)-1,3,5-triazines are, for example, 2,4,6-tris(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis(4-methylphenyl-1,3,5-triazine, 2-(2-hydroxy-4-dodecyloxyphenyl)-4,6-bis(2,4-dimethylphenyl)-1,3,5-triazine, 2-(2-hydroxy-4-tridecyloxyphenyl)-4,6-bis(2,4-dimethylphenyl)-1,3,5-triazine, 2-[2-hydroxy-4-(2-hydroxy-3-butyloxypropoxy)-phenyl]-4,6-bis(2,4-dimethyl)-1,3,5-triazine, 2-[2-hydroxy-4-(2-hydroxy-3-octyloxypropyloxy)phenyl]-4,6-bis(2,4-dimethyl)-1,3,5-triazine, 2-[4-(dodecyloxy/tridecyloxy-2-hydroxypropoxy)-2-hydroxyphenyl]-4,6-bis(2,4-dimethylphenyl)-1,3,5-triazine, 2-[2-hydroxy-4-(2-hydroxy-3-dodecyloxypropoxy)phenyl]-4,6-bis(2,4-dimethylphenyl-1,3,5-triazine, 2-(2-hydroxy-4-hexyloxy)phenyl-4,6-diphenyl-1,3,5-triazine, 2-(2-hydroxy-4-methoxyphenyl)-4,6-diphenyl-1,3,5-triazine, 2,4,6-tris[2-hydroxy-4-(3-butoxy-2-hydroxypropoxy)phenyl]-1,3,5-triazine, 2-(2-hydroxyphenyl)-4-(4-methoxyphenyl)-6-phenyl-1,3,5-triazine, 2-{2-hydroxy-4-[3-(2-ethylhexyl-1-oxy)-2-hydroxypropyloxy]phenyl}-4,6-bis(2,4-dimethylphenyl-1,3,5-triazine.

Suitable metal deactivators are, for example, N,N′-diphenyloxamide, N-salicylal-N′-salicyloylhydrazine, N,N′-bis(salicyloyl)hydrazine, N,N′-bis(3,5-di-tert-butyl hydroxyphenylpropionyl)hydrazin, 3-salicyloylamino-1,2,4-triazole, bis(benzylidene)oxalyldihydrazide, oxanilide, isophthaloyldihydrazide, sebacoyl-bis-phenylhydrazide, N,N′-diacetyladipoyldihydrazide, N,N′-bis(salicyloyl)oxylyldihydrazide, N,N′-bis(salicyloyl)thiopropionyldihydrazide.

Particularly preferred as metal deactivators are:

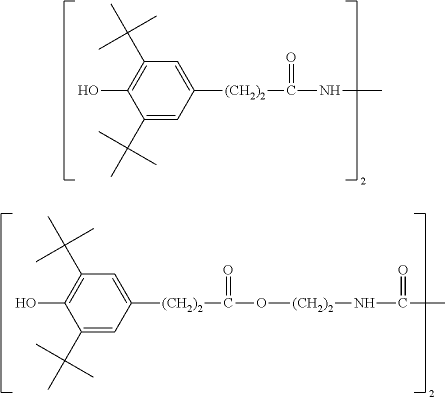

Suitable hindered amines are, for example, 1,1-bis(2,2,6,6-tetramethyl-4-piperidyl)succinate, bis(1,2,2,6,6-pentamethyl-4-piperidyl)sebacate, bis(1-octyloxy-2,2,6,6-tetramethyl-4-piperidyl)sebacate, bis(1,2,2,6,6-pentamethyl-4-piperidyl)-n-butyl-3,5-di-tert-butyl-4-hydroxybenzylmalonate, the condensation product of 1-(2-hydroxyethyl)-2,2,6,6-tetramethyl-4-hydroxypiperidine and succinic acid, linear or cyclic condensation products of N,N′-bis(2,2,6,6-tetramethyl-4-piperidyl)hexamethylenediamine and 4-tert-octylamino-2,6-dichloro-1,3,5-triazine, tris(2,2,6,6-tetramethyl-4-piperidyl)nitrilotriacetate, tetrakis(2,2,6,6-tetramethyl-4-piperidyl)-1,2,3,4-butanetetracarboxylate, 1,1′-(1,2-ethanediyl)-bis(3,3,5,5-tetramethylpiperazinone), 4-benzoyl-2,2,6,6-tetramethylpiperidine, 4-stearyloxy-2,2,6,6-tetramethylpiperidine, linear or cyclic condensation products from N,N′-bis(2,2,6,6-tetramethyl-4-piperidyl)hexamethylenediamine and 4-morpholino-2,6-dichloro-1,3,5-triazine the reaction product of 7,7,9,9-tetramethyl-2-cycloundecyl-1-oxa-3,8-diaza-4-oxospiro-[4,5]decane and epichlorohydrin.

The sterically hindered N—H, N-alkyl such as N-methyl or N-octyl, the N-alkoxy derivatives such as N-methoxy or N-octyloxy, the cycloalkyl derivatives such as N-cyclohexyloxy and the N-(2-hydroxy-2-methylpropoxy) analogs are also each included in the above-given structures here.

Preferred hindered amines also have the following structures:

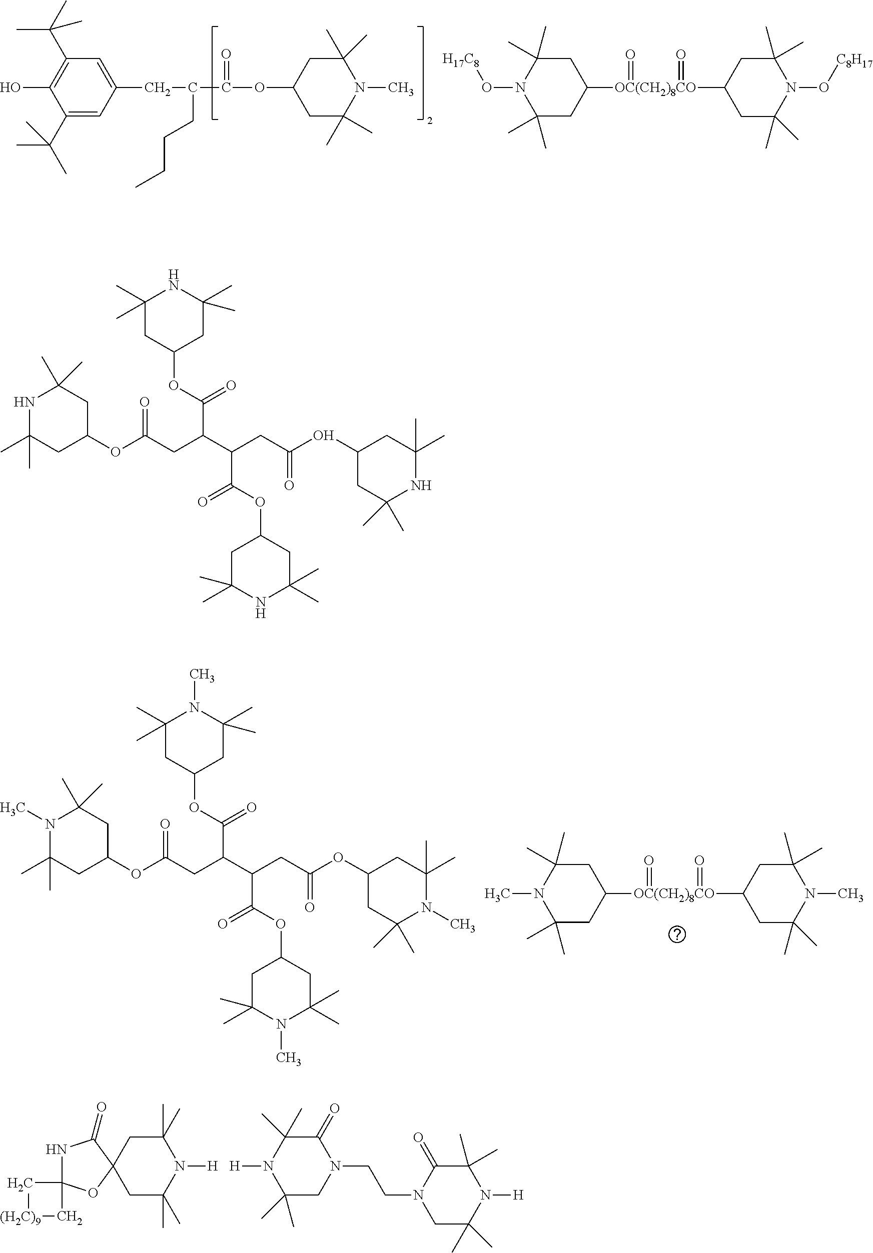

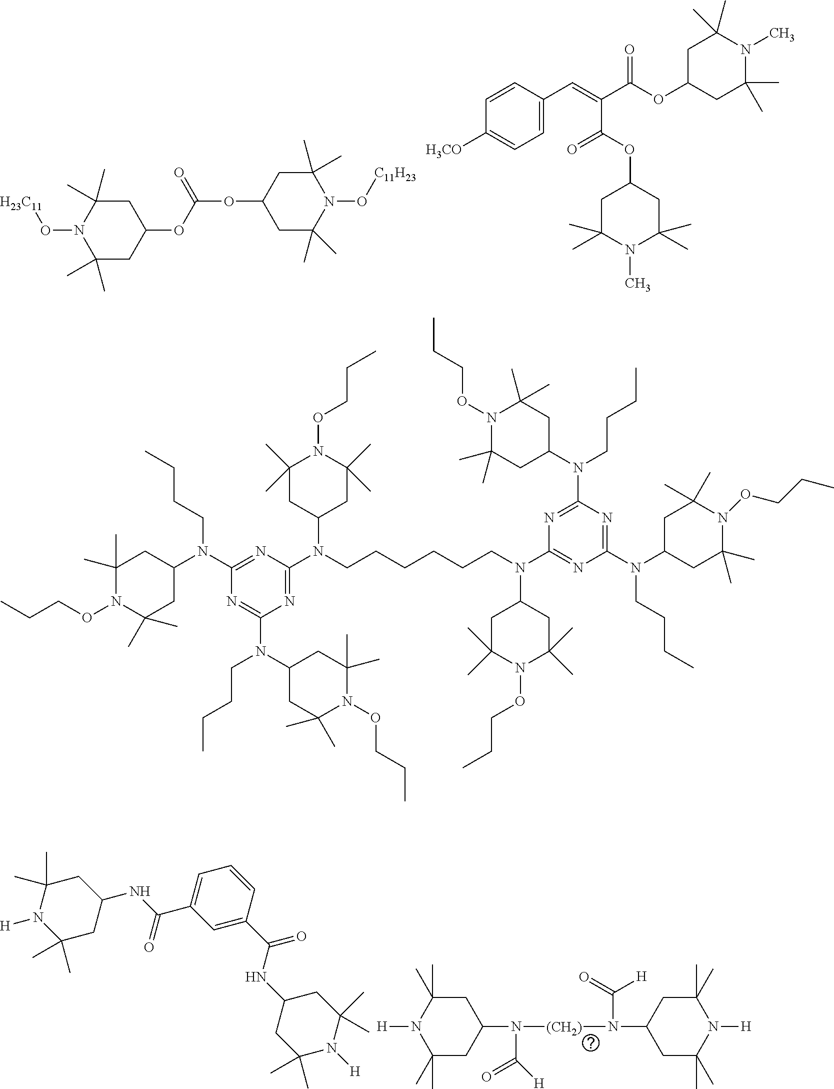

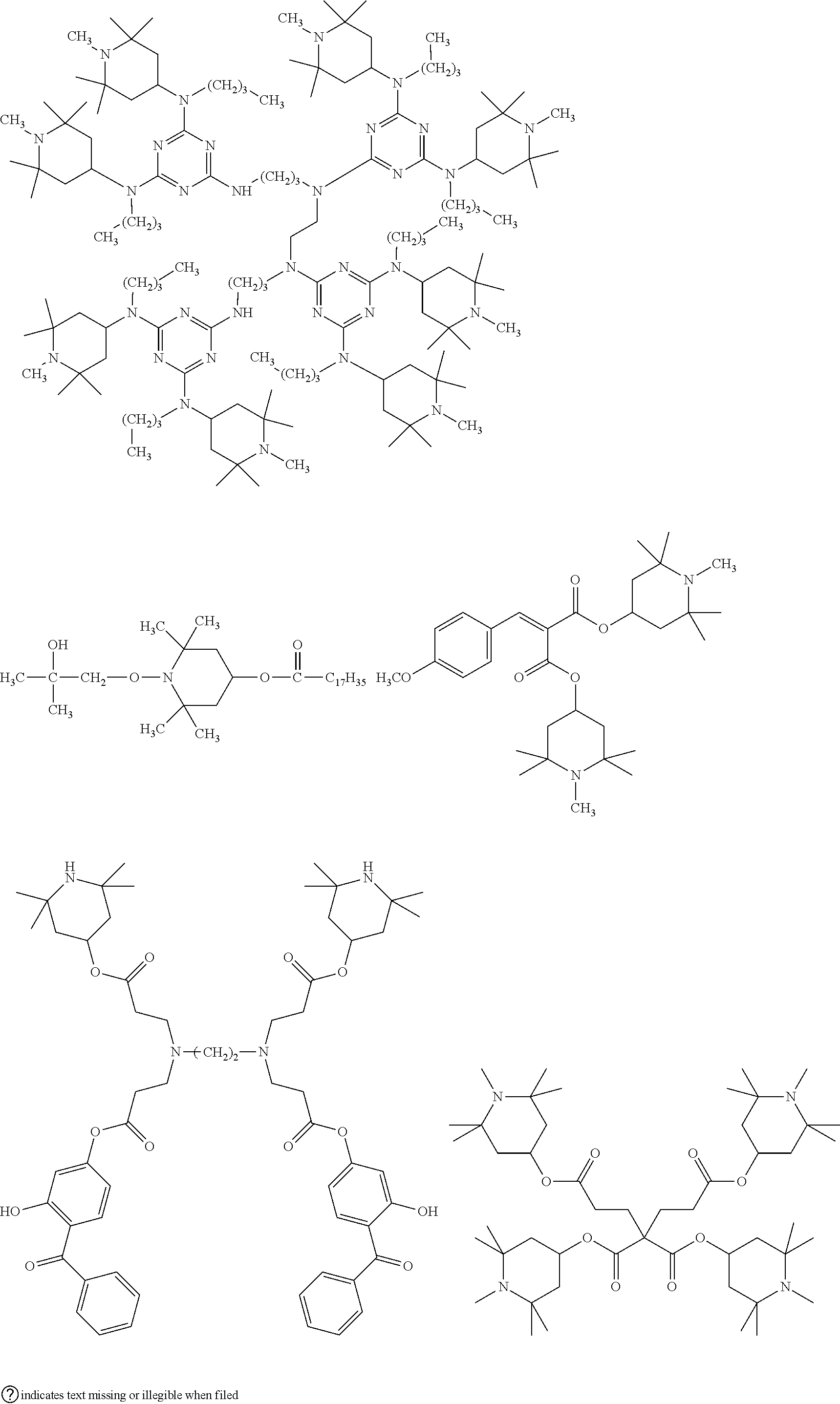

Preferred oligomeric and polymeric hindered amines have the following structures:

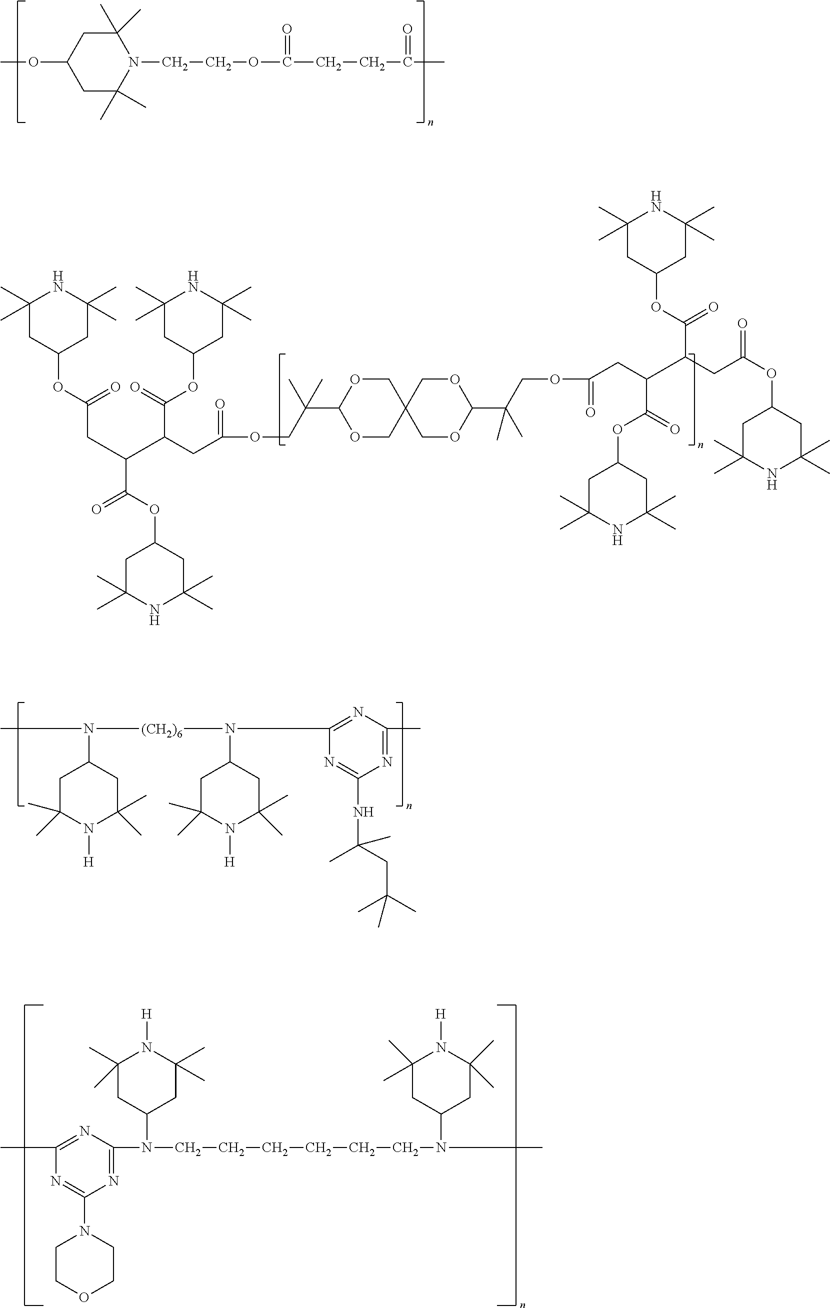

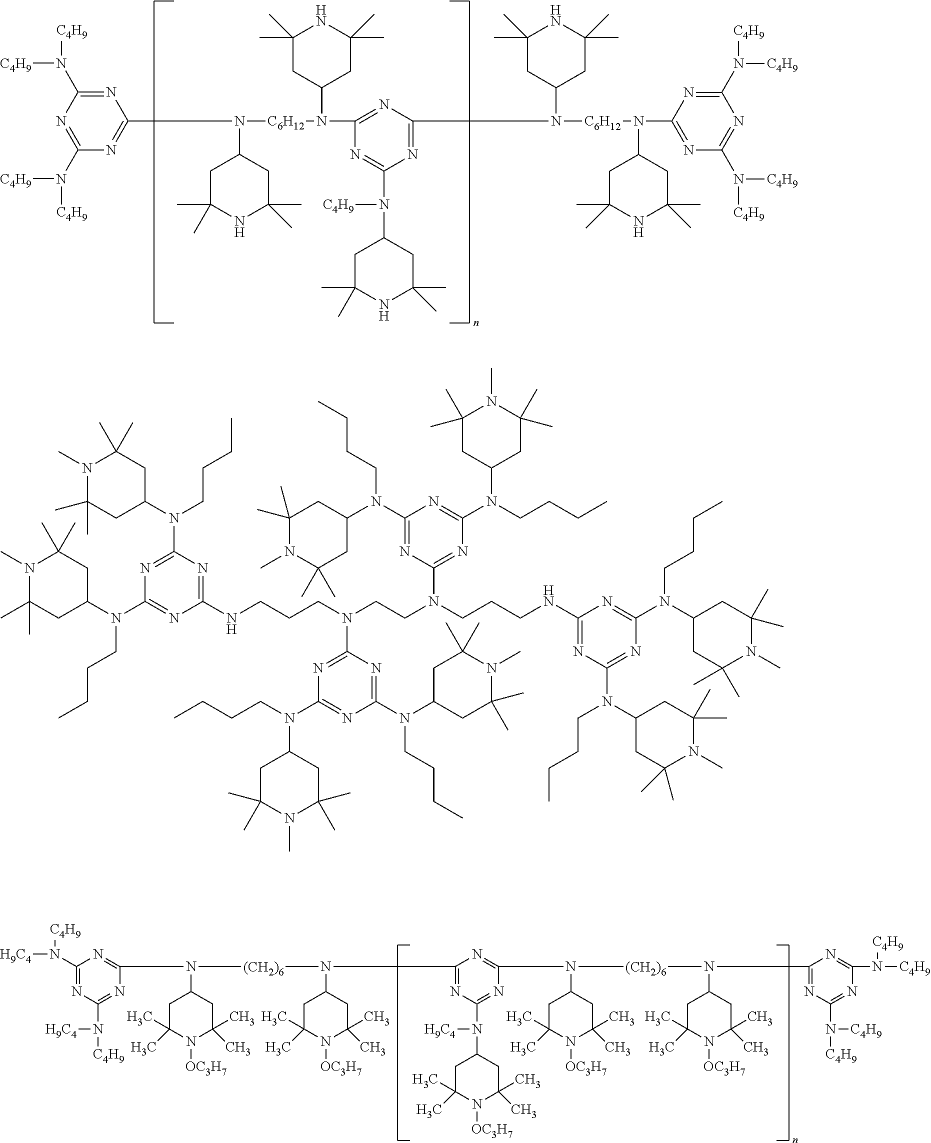

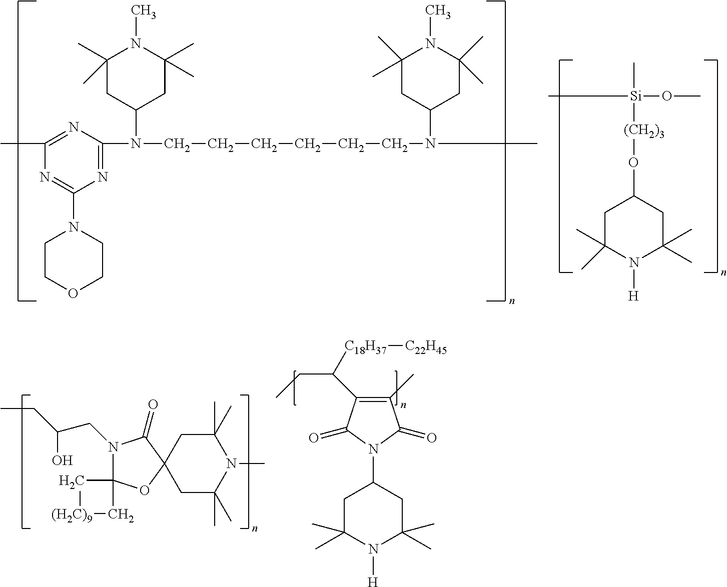

In the above-named compounds, n respectively means 3 to 100.

A further suitable light stabilizer is Hostanox NOW (manufacturer: Clariant SE) having the following general structure:

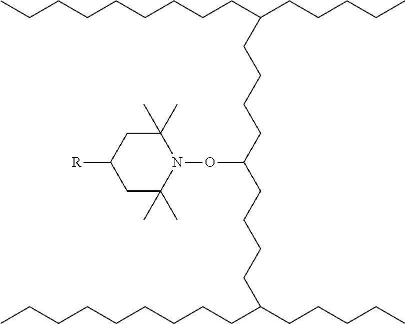

where R means —O—C(O)—C₁₅H₃₁ or —O—C(O)—C₁₇H₃₅.

Suitable dispersing agents are, for example:

polyacrylates, for example, copolymers having long chain side groups, polyacrylate block copolymers, alkylamides: for example, N,N′-1,2-ethanediylbisoctadecanamide sorbitan esters, for example, monostearyl sorbitan esters, titanates and zirconates, reactive copolymers having functional groups, for example, polypropylene-co-acrylic acid, polypropylene-co-maleic anhydride, polyethylene-co-glycidyl methacrylate, polystyrene-alt-maleic anhydride-polysiloxanes: for example, dimethylsilanediol-ethylene oxide copolymer, polyphenylsiloxane copolymer, amphiphilic copolymers: for example, polyethylene block polyethylene oxide, dendrimers, for example, dendrimers comprising hydroxyl groups.

Suitable antinucleating agents are azine dyes, such as e.g. nigrosine.

Suitable flame retardant agents are, in particular
a) inorganic flame retardant agents such as e.g. Al(OH)₃, Mg(OH)₂, AlO(OH), MgCO₃, sheet silicates such as montmorillonite or sepiolite, unmodified or organically modified double salts such as Mg—Al silicates, POSS (polyhedral oligomeric silsesquioxanes) compounds, huntite hydromagnesite or halloysite and Sb₂O₃, Sb₂O₅, MoO₃, zinc stannate, zinc hydroxystannate,
b) flameproofing agents comprising nitrogen such as melamine, melem, melam, melon, melamine derivatives, melamine condensation products or melamine salts, benzoguanamine, polyisocyanurates, allantoin, phosphacenes, in particular melamine cyanurate, melamine phosphate, dimelamine phosphate, melamine pyrophosphate, melamine polyphosphate, melamine metal phosphates such as melamine aluminum phosphate, melamine zinc phosphate, melamine magnesium phosphate, and the corresponding pyrophosphates and polyphosphates, poly-[2,4-(piperazine-1,4-yl)-6-(morpholine-4-yl)-1,3,5-triazine], ammonium polyphosphate, melamine borate, melamine hydrobromide,
c) radical formers, such as alkoxyamines, hydroxylamine esters, azo compounds, sulfene amides, sulfene imides, dicumyl or polycumyl, hydroxyimide and derivatives thereof such as e.g. hydroxyimide esters or hydroxyimide ethers,
d) phosphorus-comprising flame retardants, such as e.g. red phosphorus, phosphates, such as e.g. resorcinol diphosphate, bisphenol A diphosphate and their oligomers, triphenyl phosphate, ethylene diamine diphosphate, phosphinates, such as e.g. salts of hypophosphorous acid and their derivatives, such as e.g. alkyl phosphinate salts for example diethyl phosphinate aluminum or diethyl phosphinate zinc or aluminum phosphinate, aluminum phosphite, aluminum phosphonate, phosphonate esters, oligomeric and polymeric derivatives of methanephosphonic acid, 9,10-dihydro-9-oxa phosphorylphenanthrene-10-oxide (DOPO) and their substituted compounds,
e) halogenated flameproofing agents based on chlorine and bromine, such as e.g. polybrominated diphenyl oxides, such as e.g. decabromodiphenyloxide, tris(3-bromo-2,2-bis (bromomethyl)propyl-phosphate, tris(tribromoneopentyl)phosphate, tetrabromophthalic acid, 1,2-bis(tribromophenoxy)ethane, hexabromocyclododecane, brominated diphenylethane, tris-(2,3-dibrompropyl)isocyanurate, ethylene-bis-(tetrabromophthalimide), tetrabromo-bisphenol A, brominated polystyrene, brominated polybutadiene or polystyrene brominated polybutadiene copolymers, brominated polyphenylene ether, brominated epoxy resin, polypentabromobenzylacrylate, optionally in combination with Sb₂O₃ and/or Sb₂O₅,
f) borates, such as e.g. zinc borate or calcium borate, optionally on a carrier material, such as e.g. silica
g) sulfur-comprising compounds, such as e.g. elemental sulphur, disulfides and polysulfides, thiuram sulfide, dithiocarbamates, mercaptobenzothiazole and sulfenamides,
h) anti-drip agents, such as e.g. polytetrafluoroethylene,
i) silicon-comprising compounds, such as polyphenyl siloxanes,
j) carbon modifications, such as carbon nanotubes (CNTs), expandable graphite or graphene
k) and combinations or mixtures thereof.

Suitable plasticizers are, for example, phthalic acid esters, adipic acid esters, esters of citric acid, ester of 1,2-cyclohexane dicarboxylic acid, trimellitic acid esters, isorobide esters, phosphate esters, epoxides such as epoxidized soy bean oil, or aliphatic polyesters.

Suitable mold lubricants and processing aids are, for example, polyethylene waxes, polypropylene waxes, salts of fatty acids such as calcium stearate, zinc stearate, or salts of montane waxes, amide waxes such as erucic acid amide or oleic acid amides, fluoropolymers, silicones, or neoalkoxytitanates and zirconates.

Suitable pigments can be of an inorganic or organic nature. Inorganic pigments are, for example, titanium dioxide, zinc oxide, zinc sulfide, iron oxide, ultramarine, black carbon; organic pigments are, for example, anthraquinones, anthanthrones, benzimidazolones, chinacridones, diketoptyrrolopyrrols, dioxazines, inanthrones, isoindolines, azo compounds, perylenes, phthalocyanines or pyranthrones. Further suitable pigments are metal-based effect pigments or metal-oxide-based pearlescent pigments.

Suitable optical brighteners are, for example, bis-beznzoxazoles, phenylcumarines, or bis(styryl)biphenyls and in particular optical brighteners of the formulas:

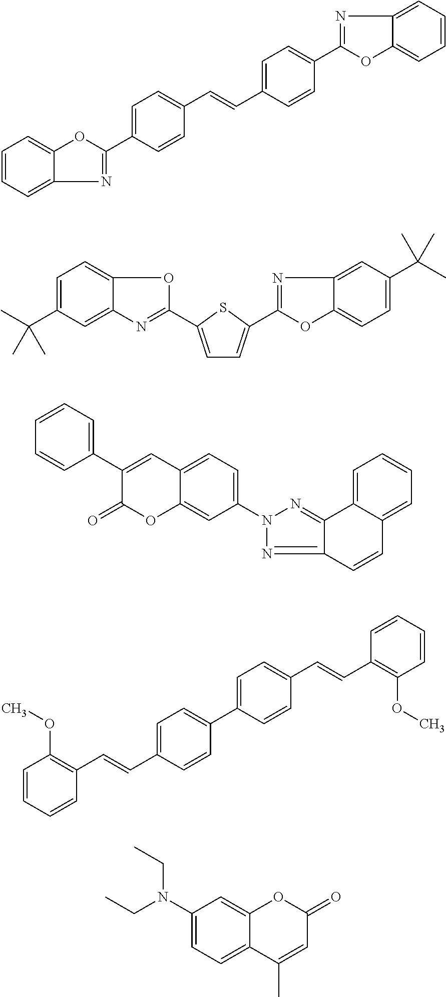

Suitable filler deactivators are, for example, polysiloxanes, polyacrylates, in particular block copolymers like polymethacrylic acid-polyalkylene oxide or polyglycidyl (meth)acrylates and their copolymers for example with styrene, and epoxides, for example, of the following structures:

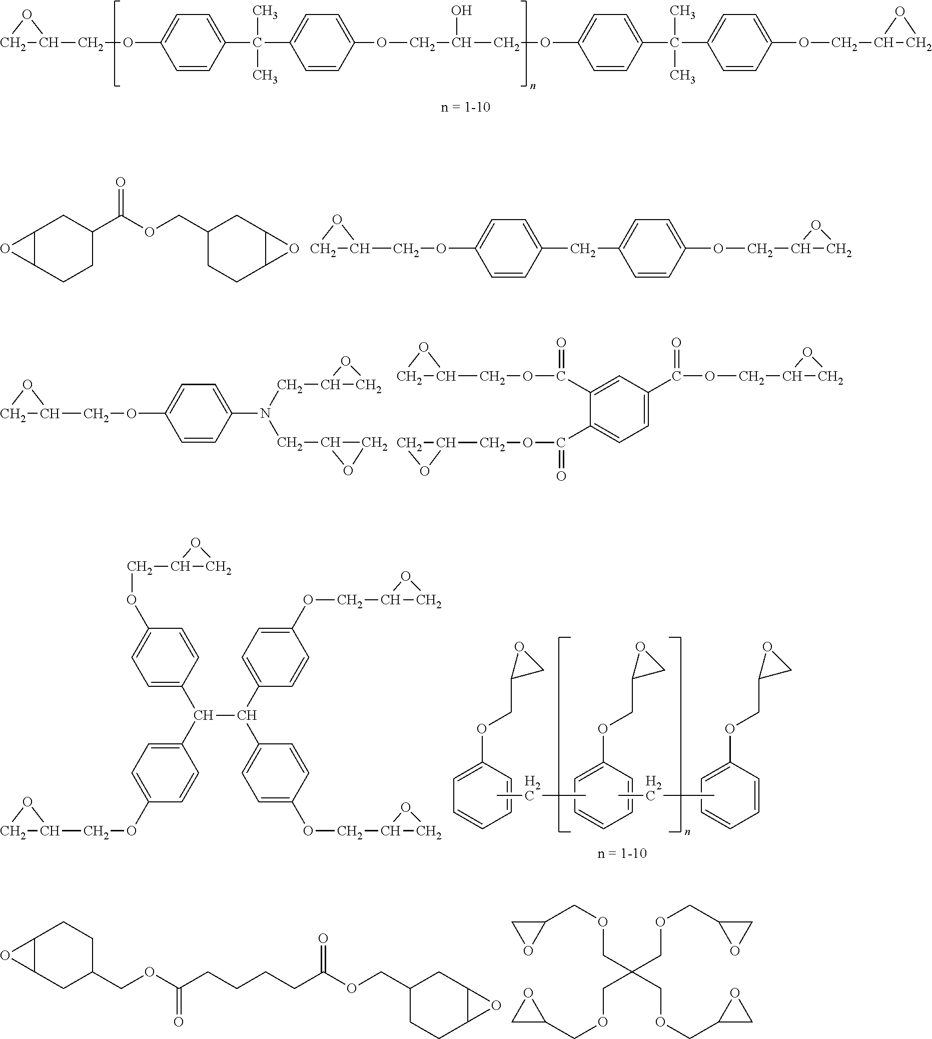

Suitable antistatic agents are, for example, ethoxylated alkylamines, fatty acid esters, alkylsulfonates, and polymers such as polyetheramides.

Suitable antiozonants are the above-mentioned amines such as N,N′-di-isopropyl-p-phenylenediamine, N,N′-di-sec-butyl-p-phenylenediamine, N,N′-bis(1,4-dimethylpentyl)-p-phenylenediamine, N,N′-dicyclohexyl-p-phenylenediamine, N-isopropyl-N′-phenyl-p-phenylenediamine, N-(1,3-dimethylbutyl)-N′-phenyl-p-phenylenediamine, N-(1-methylheptyl)-N′-phenyl-p-phenylenediamine, N-cyclohexyl-N′-phenyl-p-phenylenediamine

Suitable nucleation agents are, talcum, alkali, or alkaline earth salts of mono- and polyfunctional carboxylic acids such as benzoic acid, succinic acid, adipic acid, e.g. sodium benzoate, zinc glycerolate, aluminiumhydroxy-bis(4-tert-butyl)benzoate, 2,2′-methylene-bis-(4,6-di-tert-butylphenyl)phosphate, and trisamides and diamides such as trimesic acid tricyclohexylamide, trimesic acid tri(4-methylcyclohexylamide), trimesic acid tri(tert-butylamide), N,N′,N″-1,3,5 benzoltriyltris(2,2-dimethyl-propanamide) or 2,6-naphthalene dicarboxylic acid cyclohexylamide.

Suitable additives for increasing the molecular weight of polycondensation polymers (chain extenders) are diepoxides, bis-oxazolines, bis-oxazolones, bis-oxazines, diisocyanates, dianhydrides, bis-acyllactams, bis-maleimides, dicyanates, carbodiimides. Further suitable chain extenders are polymeric compounds, such as e.g. polystyrene-polyacrylate-polyglycidyl(meth)acrylate copolymers, polystyrene-maleic anhydride copolymers and polyethylene-maleic anhydride copolymers.

Suitable additives for increasing the electrical conductivity are, for example, the antistatic agents mentioned, carbon black and carbon compounds like carbon nanotubes and graphene, metal powder, such as e.g. copper powder, and conductive polymers, such as e.g. polypyrroles, polyanilines and polythiophenes. Suitable additives to increase thermal conductivity are, for example, aluminum nitrides and boron nitrides.

Suitable infrared-active additives are, for example, aluminum silicates or dyestuffs such as phthalocyanines or anthraquinones.

Suitable mold release agents are, for example, silicones, soaps and waxes, such as e.g. montan waxes.

Furthermore, the additives according to the invention can be used to stabilize oils, fats and chemical products. If the organic materials are oils and fats, these can be based on mineral oils, vegetable fats or animal fats or oils, fats or waxes based on, for example, synthetic esters. Vegetable oils and fats are, for example, palm oil, olive oil, rapeseed oil, linseed oil, soybean oil, sunflower oil, castor oil; animal fats are, for example, fish oils or beef tallow. The compounds according to the invention can also be used as stabilizers for lubricants, hydraulic oils, motor oils, turbine oils, gear oils, metal working fluids or as lubricating greases. These mineral or synthetic lubricants are mainly based on hydrocarbons. Chemical products are, for example, those used to stabilize polyols used in the production of polyurethanes or to stabilize monomers such as styrene, acrylic esters, methacrylic esters during transport and storage.

The present invention also relates to a method for stabilizing organic materials, in particular against oxidative, thermal and/or actinic degradation, in which a compound or a plurality of compounds according to general Formula I

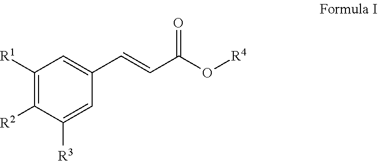

where R¹, R², R³ and R⁴ are as defined in any one of claims 1 and 5, is incorporated into the organic material.

All preferred statements relating to the compound of the general Formula I as formulated above also apply without restriction to the method according to the invention.

The invention also relates to compounds according to general Formula I

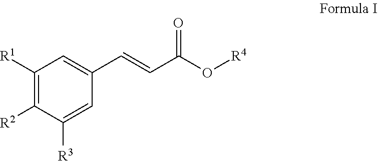

where
R¹, R² and R³ are each independently selected from the group consisting of hydroxy, linear or branched alkoxy groups having 1 to 6 carbon atoms and hydrogen, with the proviso that at least one of the moieties R¹, R² and R³ is a hydroxy moiety and/or a linear or branched alkoxy group having 1 to 6 carbon atoms, and
the following moieties according to Formulas IIa, IIb and IIc

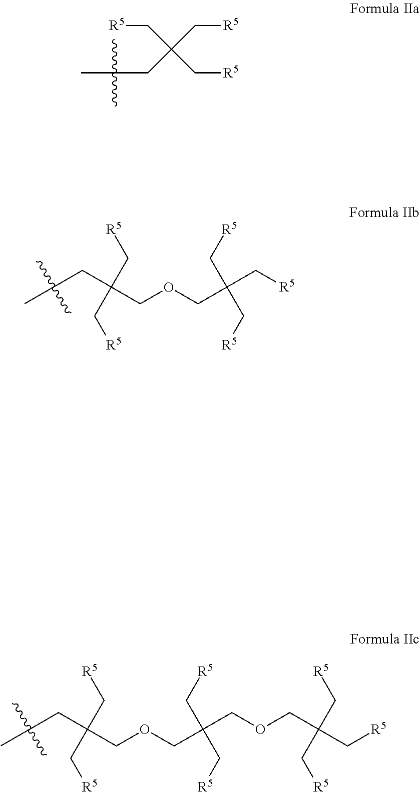

where R⁵ is the same or different at each occurrence and is selected from the group consisting of hydroxy and the following moiety according to

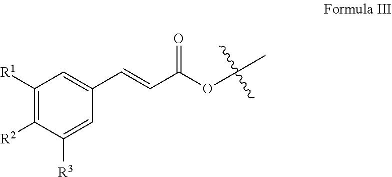

where R¹, Wand Ware as defined above.

All preferred statements relating to the moieties R¹ to R⁵ as stated above also apply without restriction to the compounds according to the invention.

The additive described above and optionally additional additives are incorporated into the plastic through conventional processing methods, wherein the polymer is melted and mixed with the additive composition according to the invention and the optionally further additives, preferably using mixers, kneaders or extruders. Extruders, such as e.g. single-screw extruders, twin-screw extruders, planetary roller extruders, ring extruders, cci-kneaders, which are preferably equipped with vacuum degassing, are preferred as processing machines. The processing can hereby take place under air or optionally under inert gas conditions such as e.g. under nitrogen.

Furthermore, the additive compositions according to the invention can be produced and introduced in the form of so-called masterbatches or concentrates, for example comprising 10-90% of the additives according to the invention in a polymer.

Areas of application according to the invention for the stabilization or organic materials according to the invention are in particular plastics in the form of injection molded parts, foils or films, foams, fibers, cables and pipes, profiles, hollow bodies, ribbons, membranes, such as e.g. geomembranes, or adhesives, which are produced via extrusion, injection molding, blow molding, calendering, pressing processes, spinning processes, rotomoulding, for example, for the electrical and electronics industry, construction industry, transport industry (car, plane, ship, train), for medical applications, for household and electrical appliances, vehicle parts, consumer goods, packaging, furniture, textiles. A further area of application are paints, colorants and coatings, and oils and fats.

The present invention will be described in more detail with reference to the following exemplary configurations without restricting the invention to the specific embodiments set out.

### EMBODIMENTS

A) Preparation of the Hydroxycinnamic Acid Salts According to the Invention

A1) Synthesis of the Methyl Ferulate

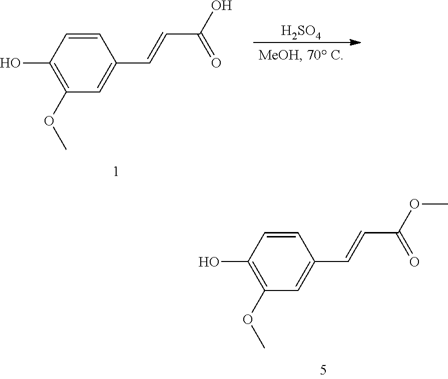

In a 500 mL round bottom flask, 15 g (1.00 eq., 77.24 mmol) ferulic acid are dissolved in 300 mL (95.77 eq., 237.00 g, 7397.00 mmol) methanol with gentle heating. Then 3.1 mL (0.75 eq., 57.99 mmol) of 95% sulfuric acid are added, the solution turning yellow. The solution is heated at 70° C. for 3 h and, after cooling, is added to 375 mL of chloroform. The solution is then washed twice with 300 mL distilled water each and once with a saturated sodium bicarbonate solution. The organic phase is dried over 100 g of sodium sulfate and the chloroform is then distilled off. After drying under high vacuum, 14.03 g (67.39 mmol) of a white solid are obtained. The yield is 87.25%.

A2) Synthesis of the Octadecyl Ferulate

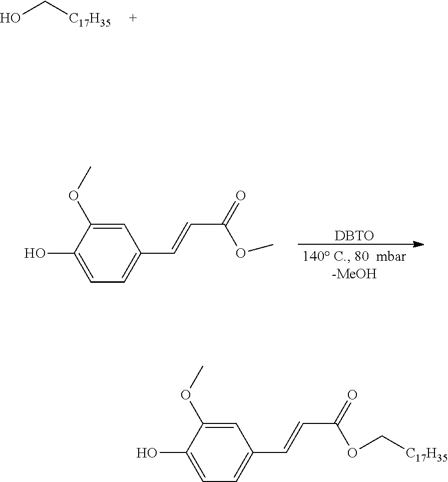

First 3.00 g (1.00 eq, 14.41 mmol) of methyl ferulate and 4.68 g (1.20 eq., 17.30 mmol) of stearyl alcohol are placed in a heated 100 mL round bottom flask fitted with a condensation bridge and cold trap in a countercurrent of nitrogen. The reaction mixture is melted at 95° C. with gentle stirring under an inert nitrogen atmosphere. 0.15 g (0.04 eq., 0.60 mmol) dibutyltin oxide (DBTO) are added to the clear melt in a nitrogen countercurrent. The reaction temperature is increased to 140° C. and a pressure of between 200-800 mbar is set. After a reaction time of 24 h, the excess stearyl alcohol is condensed off by increasing the temperature to 155° C. and reducing the pressure to 1×10-3 bar. The vacuum is broken by feeding in nitrogen and the slightly yellowish reaction melt is cooled to room temperature. This is then taken up in dichloromethane, treated with 2.80 g fuller's earth (OPTIMUM 21OFF) and refluxed for 30 min. After filtration through a short silica pad, the solvent is distilled off in vacuo. 4.11 g (9.20 mmol, 64.84%) of the white solid remain.

A3) Synthesis of the Pentaerythritol Ester of Ferulic Acid

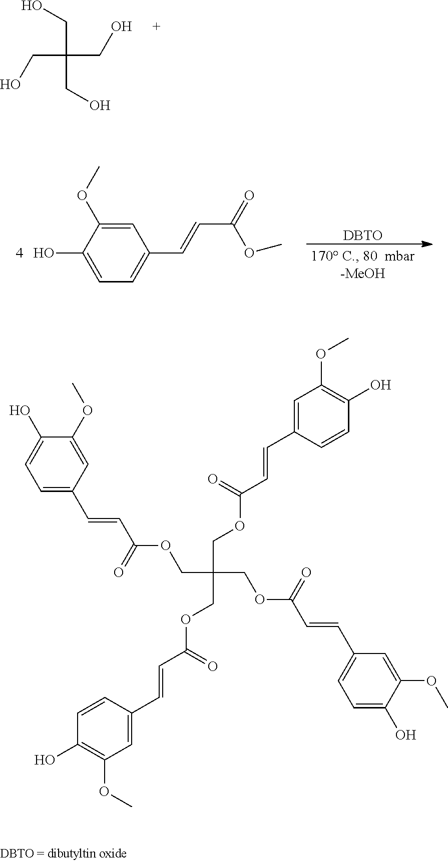 [SMILES: OCC(CO)(CO)CO ; COC(=O)/C=C/c1ccc(O)c(OC)c1 ; "DBTO" ; COc1cc(/C=C/C(=O)OCC(COC(=O)/C=C/c2ccc(O)c(OC)c2)(COC(=O)/C=C/c2ccc(O)c(OC)c2)COC(=O)/C=C/c2ccc(O)c(OC)c2)ccc1O]

2.00 g (1.00 eq., 14.69 mmol) of pentaerythritol and 12.85 g (4.20 eq., 61.76 mmol) of ferulic acid methyl ester are initially introduced in a nitrogen countercurrent into a heated 100 ml round bottom flask fitted with a condensation bridge and cold trap. The reaction mixture is melted at 100° C. with gentle stirring under an inert nitrogen atmosphere. 0.18 g (0.05 eq., 0.72 mmol) dibutyltin oxide (DBTO) are added to the clear melt in a nitrogen countercurrent. The reaction temperature is increased to 170° C. and a pressure of between 600-800 mbar is set. After a reaction time of 27 h, the excess ferulic acid methyl ester is condensed off by setting the temperature to 155° C. and reducing the pressure to 1×10-3 bar. The vacuum is broken by feeding in nitrogen and the yellow reaction melt is cooled to room temperature. This is then taken up in dichloromethane, treated with 2.80 g fuller's earth (OPTIMUM 21OFF) and refluxed for 30 min. After filtration through a short silica pad, the solvent is distilled off in vacuo. 10.02 g (11.92 mmol, 81.12%) of the finely powdered, yellow solid remain.

B) Application Check

To examine the effect of the stabilizers according to the invention, a commercial polypropylene (Moplen HP 501N, Lyondell Basell Industries) was homogenized in a powder-powder mixture with the stabilizers stated in Table and was circulated in a twin-screw microextruder (MC 5, manufacturer DSM) for 30 minutes at 200° C. and 200 revolutions per minute and the decrease in the force was recorded. The force is a direct measure for the molecular weight of polypropylene: the smaller the decrease, the higher the stabilization effect.

TABLE 1
Stabilization of polypropylene

| | | Residual strength |
|---|---|---|
| | | after 10/20/30 |
| | | minutes [%], mean |
| | Additive | values from 2 tests |
| Comparison | Without additive | 63/39/26 |
| example 1 | | |
| Example 1 in | 0.5% octadecyl | 80/72/62 |
| accordance with | ferulate | |
| the invention | | |
| Example 2 in | % pentaerythritol | 73/57/44 |
| accordance with | tetra | |
| the invention | ferulate | |
| Example 3 in | 0.25% octadecyl | 80/70/60 |
| accordance with | ferulate + | |
| the invention | 0.25% erythritol | |
| Example 4 in | 0.25% | 77/64/52 |
| accordance with | Octadecylferulate + | |
| the invention | 0.25% DSDTP | |
| Example 5 in | 0.25% pentaerythritol | 72/58/47 |
| accordance with | tetraferulate + | |
| the invention | 0.25% DSDTP | |
| Example 6 in | 0.25% octadecyl | 80/65/54 |
| accordance with | ferulate + | |
| the invention | 0.25% phosphite | |
| Example 7 in | 0.25% pentaerythritol | 80/62/42 |
| accordance with | tetraferulate + | |
| the invention | 0.25% phosphite | |

DSDTP = distearyl thiodipropionate
phosphite = Tris-(2,4-di-tert-butylphenyl)phosphite

The additives according to the invention show a clear stabilizing effect, since there is less degradation of the polymer over the course of the test.

1-15. (canceled)

16. A method of stabilizing an organic material comprising combining the organic material with a compound or mixtures of a plurality of compounds according to general Formula I 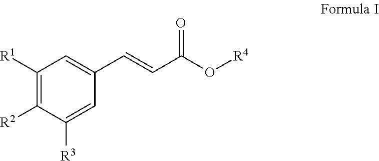
where
R¹, R² and R³ are each independently selected from the group consisting of hydroxy, linear or branched alkoxy groups having 1 to 6 carbon atoms, and hydrogen, with the proviso that at least one of the moieties R¹, R² and R³ is a hydroxy moiety and/or a linear or branched alkoxy group having 1 to 6 carbon atoms, and
R⁴ is selected from the group consisting of a linear or branched alkyl moieties having at least 8 carbon atoms and the following moieties according to Formulas IIa, IIb and IIc, 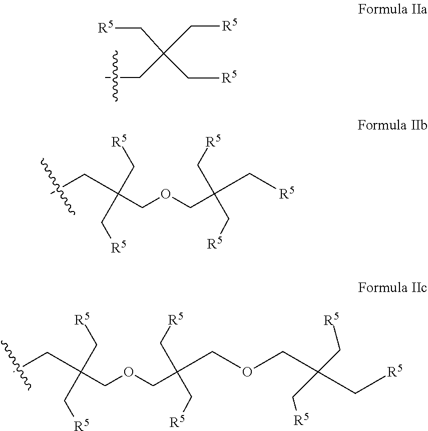
where R⁵ is the same or different at each occurrence and is selected from the group consisting of hydroxy and the following moiety according to Formula III 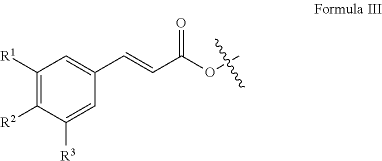
where R¹, R² and R³ are as defined above.

17. The method according to claim 16, wherein the organic material is selected from the group consisting of plastics, coatings, lubricants, hydraulic oils, engine oils, turbine oils, gear oils, metalworking fluids, chemicals, and monomers.

18. The method according to claim 16, wherein the compound according to general Formula I is selected from the following compounds:

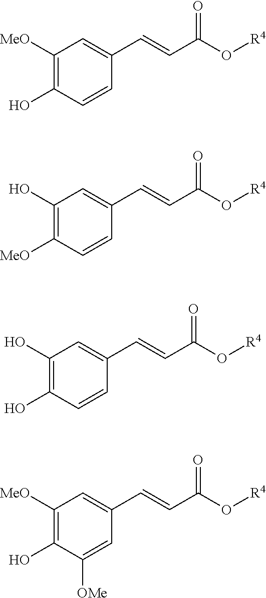

19. The method according to claim 16, wherein the linear or branched alkyl moiety having at least 8 carbon atoms of the moiety R⁴ is derived from alcohols selected from the group consisting of octan-1-ol, nonan-1-ol, decan-1-ol, undecan-1-ol, lauryl alcohol, tridecane-1-ol, myristyl alcohol, cetyl alcohol, stearyl alcohol, ceryl alcohol, myricyl alcohol, palmitoleyl alcohol, oleyl alcohol, arachidyl alcohol, behenyl alcohol, erucyl alcohol, lignoceryl alcohol, montanyl alcohol, linoleyl alcohol, isotridecyl alcohol, geraniol, rhodinool, citronellol and cerol.

20. The method according to claim 16, wherein R⁵ is selected from the following moieties

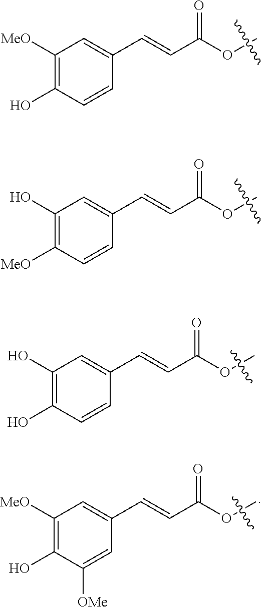

21. The method according to claim 16, wherein the substitution pattern of the moiety according to 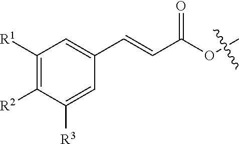
of the compound according to Formula I and the substitution pattern of the moiety R⁵ are the same.

22. The method according to claim 16, wherein the compound according to general Formula I, or in the case of a mixture of a plurality of compounds according to general Formula I, all of the compounds according to general Formula I, is contained in the organic material in a proportion by weight of 0.01 to 10.00% by weight.

23. The method according to claim 16, wherein the plastic is selected from the group consisting of
a) polymers from olefins or diolefins, natural rubber (NR), polycyclooctene, polyalkylene-carbon monoxide copolymers, copolymers of polypropylene-polyethylene (EP), EPM or EPDM with 5-ethylidene-2-norbornene as a comonomer, ethylene-vinyl acetate (EVA}, ethylene acrylic esters, ethylene acrylic acid glycidyl(meth)acrylate, polypropylene graft maleic anhydride, polypropylene graft acrylic acid, polyethylene graft-acrylic acid, polyethylene-polybutylacrylate-graft-maleic anhydride, and long-chain branched polypropylene copolymers which are produced with alpha-olefins as comonomers,
b) polystyrene, polymethylstyrene, poly-alpha-methyl styrene, polyvinylnaphthalene, polyvinylbiphenyl, polyvinyltoluene, styrene-butadiene (SB), styrene-butadiene-styrene (SBS), styrene-ethylene-butylene-styrene (SEBS), styrene-ethylene-propylene-styrene, styrene-isoprene, styrene-isoprene-styrene (SIS), styrene-butadiene-acrylonitrile (ABS), styrene-acrylonitrile (SAN), styrene-acrylonitrile-acrylate (ASA), styrene-ethylene, styrene-maleic anhydride polymers, graft copolymers made of methyl methacrylate, styrene-butadiene and ABS (MABS), and hydrogenated polystyrene derivatives,
c) halogen-containing polymers,
d) polymers of unsaturated esters, polyacrylonitrile, polyacrylamide, and copolymers of acrylonitrile and alkyl acrylate,
e) polymers of unsaturated alcohols and derivatives,
f) polyacetals and blends with polystyrene or polyamides,
g) polymers of cyclic ethers,
h) polyurethanes and polyureas,
i) polyamides, blends of different polyamides and blends of polyamides with polyolefins,
j) polyimides, polyamide-imides, polyetherimides, polyesterimides, poly(ether)ketones, polysulfones, polyethersulfones, polyarylsulfones, polyphenylene sulfides, polybenzimidazoles, and polyhydantoins,
k) polyesters made from aliphatic or aromatic dicarboxylic acids and diols or from hydroxycarboxylic acids,
l) polycarbonates, polyester carbonates, and blends,
m) cellulose derivatives,
n) epoxy resins,
o) phenolic resins,
p) unsaturated polyester resins from unsaturated dicarboxylic acids and diols with vinyl compounds,
q) silicones, and
r) mixtures, combinations or blends of two or more of the aforementioned polymers.

24. The method according to claim 16, wherein the plastic comprises at least one additional additive selected from the group consisting of primary and/or secondary antioxidants.

25. The method according claim 24, wherein the primary and/or secondary antioxidants are selected from the group consisting of phosphites, phosphonites, thiols, phenolic antioxidants, sterically hindered amines, hydroxylamines and mixtures or combinations thereof, UV absorbers, light stabilizers, hydroxylamine-based stabilizers, benzofuranone-based stabilizers, nucleating agents, impact modifiers, plasticizers, lubricants, rheology modifiers, chain extenders, processing aids, pigments, dyes, optical brighteners, antimicrobial agents, antistatic agents, slip agents, antiblocking agents, coupling agents, dispersants, compatibilizers, oxygen scavengers, acid scavengers, co-stabilizers, marking agents, and anti-fogging agents.

26. The method according to claim 24, wherein the at least one additional additive is contained or added in an amount of 0.01 to 9.99% by weight based on the total of the at least one compound according to Formula I, the organic material, and the at least one additive.

27. A stabilized organic material comprising at least one compound according to general Formula I, or a mixture of a plurality of compounds according to general Formula I, as a stabilizer 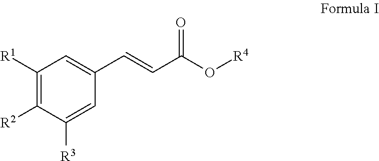
wherein R¹, R² and R³ are each independently selected from the group consisting of hydroxy, linear or branched alkoxy groups having 1 to 6 carbon atoms, and hydrogen, with the proviso that at least one of the moieties R¹, R² and R³ is a hydroxy moiety and/or a linear or branched alkoxy group having 1 to 6 carbon atoms, and
R⁴ is selected from the group consisting of a linear or branched alkyl moieties having at least 8 carbon atoms and the following moieties according to Formulas IIa, IIb and IIc, 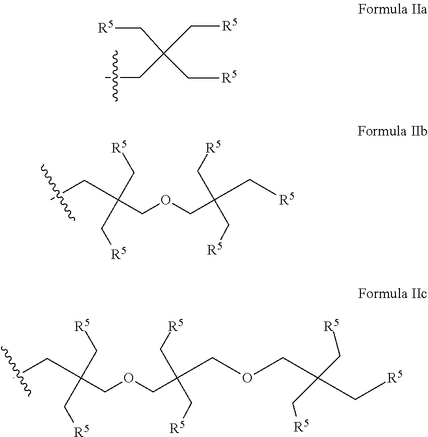
where R⁵ is the same or different at each occurrence and is selected from the group consisting of hydroxy and the following moiety according to Formula III 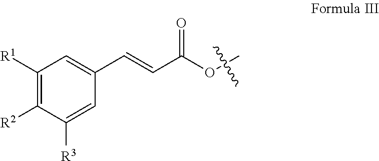
where R¹, R² and R³ are as defined above.

28. The organic material according claim 27, having the following composition:
0.01 to 10.00% by weight of a compound according to general Formula I, or, in the case of a mixture of a plurality of compounds according to general Formula I, the entirety of all compounds according to general Formula I,
99.99 to 90.00% by weight of at least one organic material, and
0 to 9.99% by weight of at least one additive,
the components adding up to 100% by weight.

29. The organic material according to claim 28, wherein the at least one additive is selected from the group consisting of primary and/or secondary antioxidants, UV absorbers, light stabilizers, hydroxylamine-based stabilizers, benzofuranone-based stabilizers, nucleating agents, impact modifiers, plasticizers, lubricants, rheology modifiers, chain extenders, processing aids, pigments, dyes, optical brighteners, antimicrobial agents, antistatic agents, slip agents, antiblocking agents, coupling agents, dispersants, compatibilizers, oxygen scavengers, acid scavengers, co-stabilizers, marking agents and anti-fogging agents.

30. A compound according to the general Formula I 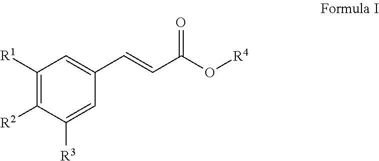
where
R¹, R² and R³ are each independently selected from the group consisting of hydroxy, linear or branched alkoxy groups having 1 to 6 carbon atoms and hydrogen, with the proviso that at least one of the moieties R¹, R² and R³ is a hydroxy moiety and/or a linear or branched alkoxy group having 1 to 6 carbon atoms, and
R⁴ is selected from the group consisting of the following moieties according to Formulas IIa, IIb and IIc 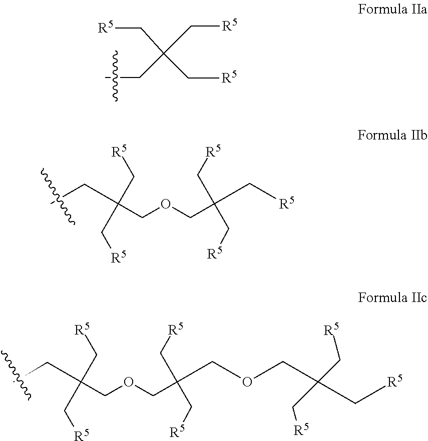
where R⁵ is the same or different at each occurrence and is selected from the group consisting of hydroxy and the following moiety according to Formula III 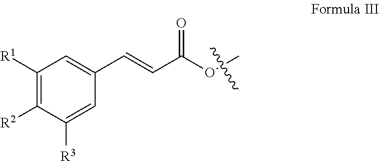
wherein R¹, R² and R³ are as defined above.
